(19) United States
(12) Patent Application Publication
(10) Pub. No.: US 2019/0328238 A1
(43) Pub. Date: Oct. 31, 2019

(54) DIGITALLY ENHANCED AND STIMULATED THERMAL IMAGING

(71) Applicant: Washington University, St .Louis, MO (US)

(72) Inventors: Samuel Achilefu, St. Louis, MO (US); Clyde George Bethea, Franklin Park, NJ (US); Cheryl Lynn Bethea, Franklin Park, NJ (US); Suman B. Mondal, St. Louis, MO (US); Hongyu Meng, St. Louis, MO (US); Christine O'Brien, St. Louis, MO (US)

(21) Appl. No.: 16/399,595

(22) Filed: Apr. 30, 2019

Related U.S. Application Data
(60) Provisional application No. 62/664,606, filed on Apr. 30, 2018.

(51) Int. Cl. A61B 5/01; H04N 5/33; G06T 11/20; G06T 7/00; A61N 7/02; A61F 7/03; A61N 5/06; A61N 5/10

(52) U.S. Cl. CPC A61B 5/015; H04N 5/33; G06T 11/206; G06T 7/0012; A61N 2005/067; A61F 7/03; A61N 5/0625; A61N 5/10; G06T 2207/30004; A61N 7/02

(57) ABSTRACT

A computer-implemented method for classifying a tissue type within a tissue sample using laser-stimulated thermal imaging (LSTI) is disclosed. The method includes obtaining a first thermal image of the tissue sample, introducing at least one thermal stimulation into at least one thermal stimulation volume within the tissue sample, obtaining a second thermal image of the tissue sample, creating a spatial temperature plot based on the first and second thermal images, determining at least one thermal diffusion parameter from the spatial temperature plot, and classifying each tissue type adjacent to each of the at least one thermal stimulation volumes based on the at least one thermal diffusion parameter.

### BACKGROUND

The diagnosis of pathophysiological conditions, such as cancerous tissues, is an ongoing challenge in medicine. Existing methods of diagnosis and treatment monitoring may include so many separate tests and events as to discourage a patient from pursuing diagnosis until a pathophysiological condition is so advanced as to be apparent, and consequently more challenging to treat.

Depending upon the type of disease, diagnosis is typically performed in a multi-step process beginning with either visual inspection by a trained medical provider or a non-invasive imaging method, followed by a biopsy of tissue identified as suspicious for the presence of disease. This process may require repeated visits to a medical provider, which may not be practical for many patients, in particular low income subjects. Moreover, histopathologic evaluation may require a large number of biopsies, the majority of which are typically benign. Biopsies cause a great deal of anxiety for patients and increase patient costs, and the process is typically time-consuming for medical providers and pathologists. As a result, many patients only comply with the current procedure when the disease causes significant symptoms, leading to late diagnosis and therefore poor patient outcomes.

Improved non-invasive imaging systems with sufficiently high sensitivity and specificity may significantly reduce the number of biopsies performed and potentially obviate their use in certain applications. In addition, the development of suitably portable non-invasive imaging systems may enable screening of patients that are typically not reached due to the cost of health care or lack of access to high quality medical facilities, such as patients living in rural areas. In addition, these improved non-invasive imaging systems may enable “see-and-treat” approaches by local practitioners, in which a patient may receive treatment the same day, thereby preventing patient loss to follow-up. Trials where see-and-treat strategies are employed have reported significantly higher patient compliance and most importantly, improved patient outcomes.

Thermal imaging has long been used as an adjunct clinical imaging modality for cancer diagnosis based on observed steady-state temperature differences between tumors and healthy tissue due to increased blood flow and metabolic activity. Advances in the field of thermal imaging, including improvements to detector sensitivity and high spatiotemporal resolution, have enhanced the ability of thermal imaging to identify tumor tissues. Recently, dynamic thermal imaging has been developed to further enhance the capabilities of thermal contrast imaging by challenging tissues of interest with a colder temperature and tracking thermal recovery of the cooled tissue to body temperature, with a faster rate of recovery observed in cancerous regions. Although thermally challenging tissues during thermal imaging enables higher sensitivity than conventional steady state thermal imaging, dynamic thermal imaging at present remains unable to detect tumors located more than a few millimeters below the skin's surface because of the limited penetration depth of topically administered cold challenges. Moreover, the discomfort of applying freezing temperatures to a patient's skin and the difficulty in developing a standardized protocol for cooling a patient's skin for a thermal challenge pose additional challenges to the implementation of dynamic thermal imaging.

### BRIEF DESCRIPTION OF THE DISCLOSURE

In one aspect, a computer-implemented method for classifying a tissue type within a tissue sample using a laser-stimulated thermal imaging (LSTI) device is provided. The LSTI device is communicatively coupled to a processor and a memory. The method includes obtaining a first thermal image of the tissue sample, and introducing at least one thermal stimulation into at least one thermal stimulation volume within the tissue sample. The method also includes obtaining a second thermal image of the tissue sample, and creating a spatial temperature plot based on the first and second thermal images. The method further includes determining at least one thermal diffusion parameter from the spatial temperature plot, and classifying each tissue type adjacent to each of the at least one thermal stimulation volumes based on the at least one thermal diffusion parameter.

In another aspect, a laser-stimulated thermal imaging (LSTI) system for classifying a tissue type within a tissue sample is provided. The system includes a LSTI device communicatively coupled to a processor and a memory. The processor of the LSTI system is configured to obtain a first thermal image of the tissue sample, and introduce at least one thermal stimulation into at least one thermal stimulation volume within the tissue sample. The processor is also configured to obtain a second thermal image of the tissue sample, and create a spatial temperature plot based on the first and second thermal images. The processor is further configured to determine at least one thermal diffusion parameter from the spatial temperature plot, and classify each tissue type adjacent to each of the at least one thermal stimulation volumes based on the at least one thermal diffusion parameter.

In yet another aspect, a non-transitory computer-readable storage medium having computer-executable instructions embodied thereon is provided. When executed by a laser-stimulated thermal imaging (LSTI) system comprising a LSTI device communicatively coupled to a processor and a memory, the computer-executable instructions cause the processor of the LSTI system to obtain a first thermal image of the tissue sample, and introduce at least one thermal stimulation into at least one thermal stimulation volume within the tissue sample. The computer-executable instructions also cause the processor to obtain a second thermal image of the tissue sample, and create a spatial temperature plot based on the first and second thermal images. The computer-executable instructions further cause the processor to determine at least one thermal diffusion parameter from the spatial temperature plot, and classify each tissue type adjacent to each of the at least one thermal stimulation volumes based on the at least one thermal diffusion parameter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A contains a laser-stimulated thermal image of a mouse tumor in a mouse's mammary fat pad in accordance with one aspect of the disclosure;

FIG. 1B is an image showing a zoomed-in view of the laser-stimulated thermal image of FIG. 1A within the region delineated by the white square in accordance with one aspect of the disclosure;

FIG. 2 contains an in vivo thermal image of the tumor (delineated with an ellipse) revealing colder temperature compared to the surrounding tissue in accordance with one aspect of the disclosure;

FIG. 3A is an image showing a 3D contour of a standard Gaussian point-spread function generated using Matlab in accordance with one aspect of the disclosure;

FIG. 3B is an image showing a simulated contour of a heat diffusion curve for healthy tissue in which the contour resembles the Gaussian contour of FIG. 3A in accordance with one aspect of the disclosure;

FIG. 3C is an image showing a simulated contour of a heat diffusion curve for tumor tissue in which the contour does not resemble the Gaussian contour of FIG. 3A in accordance with one aspect of the disclosure;

FIG. 4 is an image showing heating profiles obtained from tumor tissue (lower left spot) and normal tissue (upper right spot) in accordance with one aspect of the disclosure;

FIG. 5 contains a series of thermal images of a C57B6 mouse injected with PyMT cells in the mammary fat pad showing tumor growth progression from day 5-22 post-injection (arrows delineate tumor location) in accordance with one aspect of the disclosure;

FIG. 6A is an image showing a thermal image and corresponding heat diffusion profiles obtained from tumor tissue 19 days after injection of a C57B6 mouse's mammary fat pad with PyMT cells in accordance with one aspect of the disclosure;

FIG. 6B is an image showing a thermal image and corresponding heat diffusion profiles obtained from normal tissue of a C57B6 mouse's mammary fat pad in accordance with one aspect of the disclosure;

FIG. 7 is an image showing laser stimulated digitally enhanced thermal images of three different tumors displaying deviations from the Gaussian contour shown in FIG. 3A and FIG. 3B in accordance with one aspect of the disclosure;

FIG. 8 is an image showing the penetration depth of laser pulses with different wavelengths into biological tissue in accordance with one aspect of the disclosure;

FIG. 9 is an image showing a handheld thermal camera in accordance with one aspect of the disclosure;

FIG. 10 is an image showing thermal goggles that incorporate a small thermal camera in accordance with one aspect of the disclosure;

FIG. 11A is a schematic illustration showing the elements of a digital enhanced and LSTI thermal imaging system that includes the handheld thermal camera of FIG. 9 operatively coupled to a portable computing device in accordance with one aspect of the disclosure;

FIG. 11B is a schematic illustration showing the elements of a digital enhanced and LSTI thermal imaging system that includes the thermal goggles of FIG. 10 operatively coupled to a portable computing device in accordance with one aspect of the disclosure;

FIG. 12 is a flow chart illustrating a method for laser-stimulated thermal imaging in accordance with one aspect of the disclosure;

FIG. 13 is a block diagram showing elements of a computing system used for tracking a shape of an object depicted in a video in accordance with one aspect of the disclosure;

FIG. 14 is a block diagram of components of a computing device for use in the computing system shown in FIG. 13 in accordance with one aspect of the disclosure;

FIG. 15 is a block diagram illustrating an arrangement of components of a user computing device for use in the computing system shown in FIG. 13 in accordance with one aspect of the disclosure; and

FIG. 16 illustrates is a block diagram illustrating an arrangement of components of a server system for use in the system shown in FIG. 13 in accordance with one aspect of the disclosure;

FIG. 17 is a schematic illustration showing the elements of an automated LSTI system in accordance with one aspect of the disclosure;

FIG. 18A illustrates a top view and side view model based on Penne' s Bioheat Equation in accordance with one aspect of the disclosure;

FIG. 18B illustrates a plot of surface temperature cross-section as shown in FIG. 18A in accordance with one aspect of the disclosure;

FIG. 19 illustrates thermal curve and amplitude comparisons between computational simulation model results tested against experimental results of bacon 5× muscle and 5× fat locations in accordance with one aspect of the disclosure;

FIG. 20 illustrates in vivo LSTI from rat breast cancer tumors in accordance with one aspect of the disclosure;

FIG. 21 illustrates thermal biopsy images in accordance with one aspect of the disclosure;

FIG. 22 illustrates clinic-ready image processing pipeline procedures in accordance with one aspect of the disclosure.

Advantages will become more apparent to those skilled in the art from the following description of the preferred aspects which have been shown and described by way of illustration. As will be realized, the present aspects may be capable of other and different aspects, and their details are capable of modification in various respects. Accordingly, the drawings and description are to be regarded as illustrative in nature and not as restrictive.

### DETAILED DESCRIPTION OF THE DISCLOSURE

In various aspects, systems, devices and methods for digitally enhancing thermal imaging and laser-stimulated thermal imaging (LSTI) images to provide diagnostic information and elucidate mechanistic events in pathophysiologic conditions are disclosed below. The disclosed systems, devices, and methods enable improved accuracy of detection for cancers and other diseases using real-time dynamic thermal imaging relative to existing dynamic thermal imaging systems that make use of local thermal stimulation of tissues to assess real-time thermal mass transport in tumors and the uninvolved tissues. In addition, the disclosed systems, devices, and methods are capable of obtaining both macroscopic and microscopic images at resolutions up to a 10 μm spatial diffraction limit. In various aspects, the disclosed systems, devices, and methods are suitable for interrogating normal physiology and pathophysiology of various tissues. By way of non-limiting example, the disclosed systems, devices, and methods enable the detection of lesions such as cancer in real-time. The digitally enhanced thermal images obtained using the disclosed systems, devices, and methods offer improved resolution and contrast over standard thermal images. As disclosed herein, the systems, devices, and methods are capable of detecting diverse sets of diseases, monitoring normal or pathophysiological conditions, aiding in medical interventions, monitoring treatment responses, and providing real-time screening information at point-of-care settings.

In one aspect, the disclosed systems, devices, and methods obtain laser-stimulated thermal images by delivering a point source of heat into tissue via a heat source including, but not limited to, a low powered laser, observing the resulting thermal diffusion profile using a thermal imaging device, and identifying cancerous versus healthy tissue based on one or more characteristics of the thermal diffusion profile. In contrast to the cold challenge technique, this strategy can be used to stimulate both superficial and deep tumors by varying the laser wavelength, duration, and power to target the position of heat delivery and to modulate the amount of heat delivered to the targeted position. Further, the use of a laser for heat delivery to the tissues enables the completed of thermal tissue stimulation within seconds rather than the several minutes necessary to cool tissues in existing dynamic thermal imaging methods.

By measuring the heat diffusion profile in the vicinity of the point heat source, the disclosed LSTI method enables the capture of the differential thermal response of healthy and diseased tissues using parameters including, but not limited to, thermal peak width, thermal peak height, attenuation, symmetry, and field inhomogeneity with high spatial resolution, providing both macroscopic and microscopic information simultaneously.

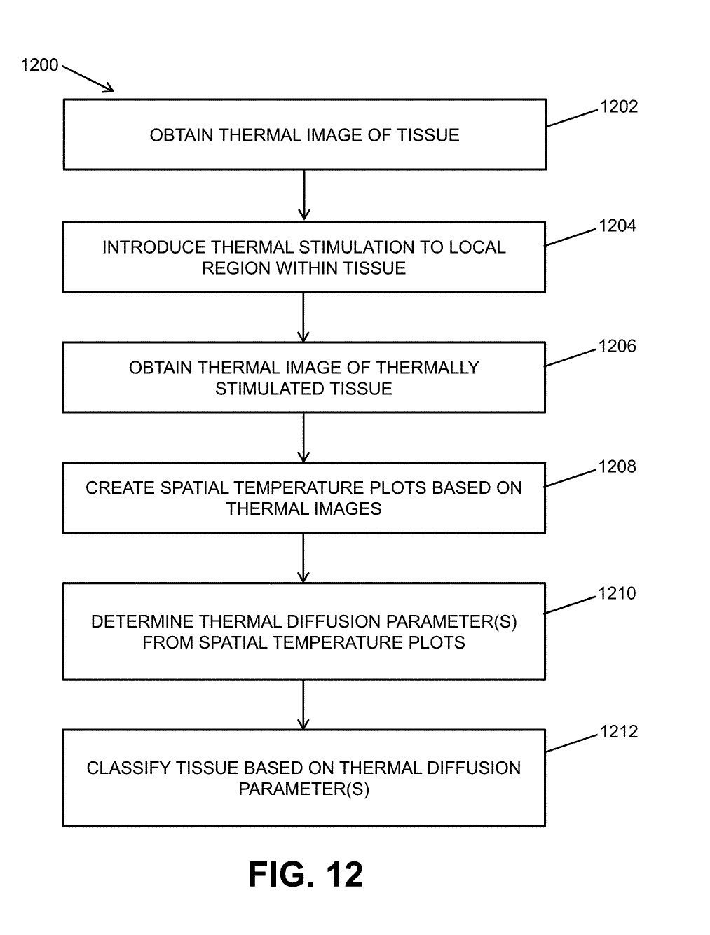

FIG. 12 is a flow chart illustrating a laser-stimulated thermal imaging method 1200 in one aspect. As illustrated in FIG. 12, the method 1200 includes obtaining a thermal image of the tissue prior to any thermal stimulation at 1202. Without being limited to any particular theory, the thermal image obtained at 1202 may serve as a baseline from which changes in the spatial distribution of temperatures induced by thermal stimulation may be assessed. The thermal image may be obtained using any suitable thermal imaging device or sensor without limitation. Non-limiting examples of suitable thermal imaging devices and sensors include thermally sensitive cameras, temperature sensors, photon sensors, and any other suitable devices or sensors sensitive to temperature, heat, or radiation variations. In various aspects, the thermal images may include macroscopic and microscopic thermal images characterized by resolutions as small as a 10 μm spatial diffraction limit.

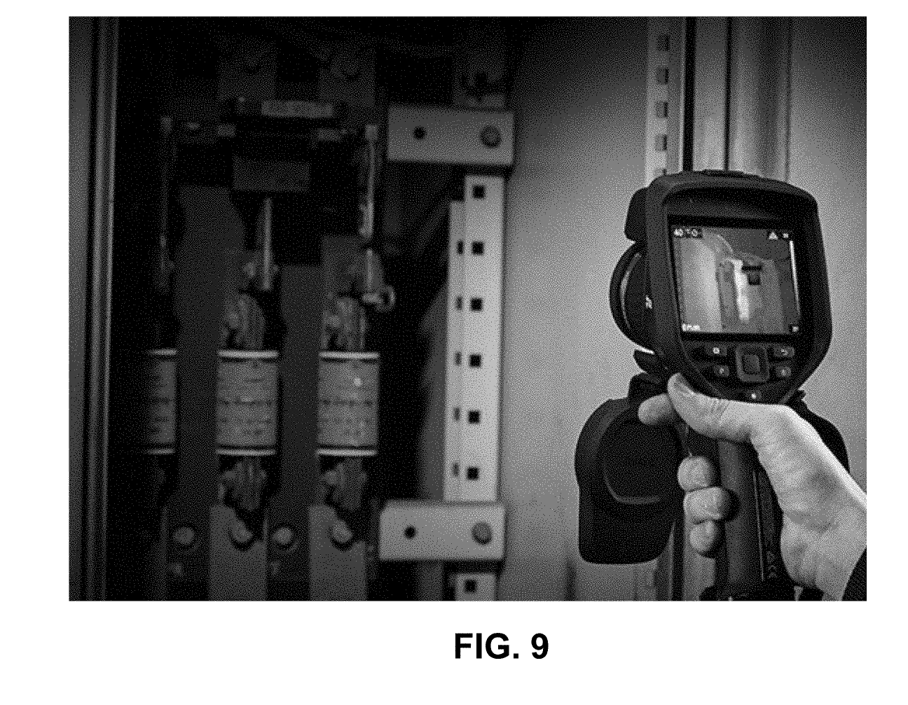

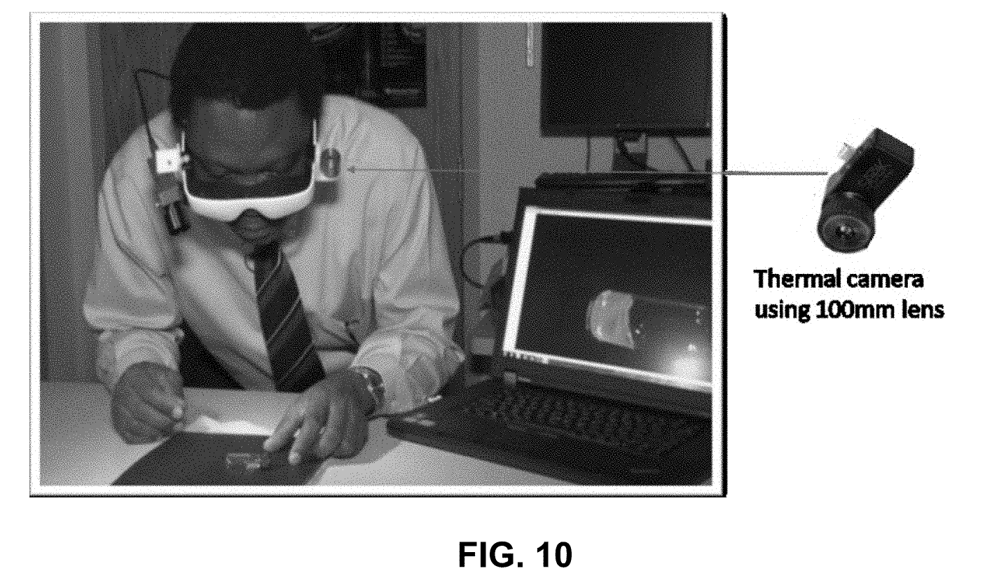

In various other aspects, the thermal imaging device may be provided in any form compatible with the intended environment of use. In one aspect, the thermal imaging device may include a fixed thermally sensitive camera, microscope, or thermal sensor, including, but not limited to a fixed thermally sensitive camera mounted to a fixed support. Non-limiting examples of suitable fixed supports to which a fixed thermal imaging device may be mounted include a tripod, a stand, a gantry arm, and any other suitable fixed support without limitation. In another aspect, the thermal imaging device may be provided in the form of a portable or hand-held device. By way of example, the thermal imaging device may be a hand-held thermal imaging device, as illustrated in FIG. 9. In another additional aspect, the thermal imaging device may be provided in the form of a wearable device including, but not limited to, thermal imaging goggles, as illustrated in FIG. 10. The thermal imaging goggles may further enable multiplexed display of data from additional imaging modalities including, but not limited to,

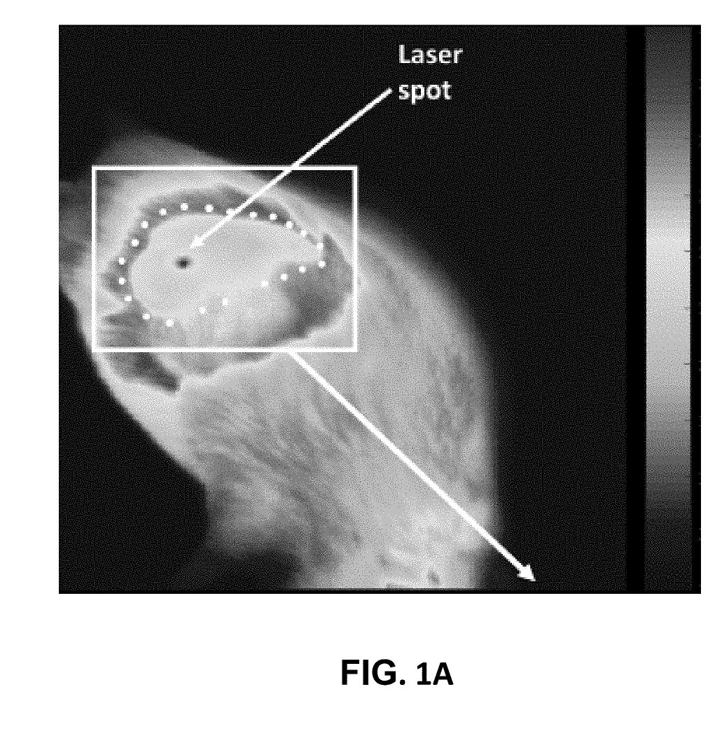

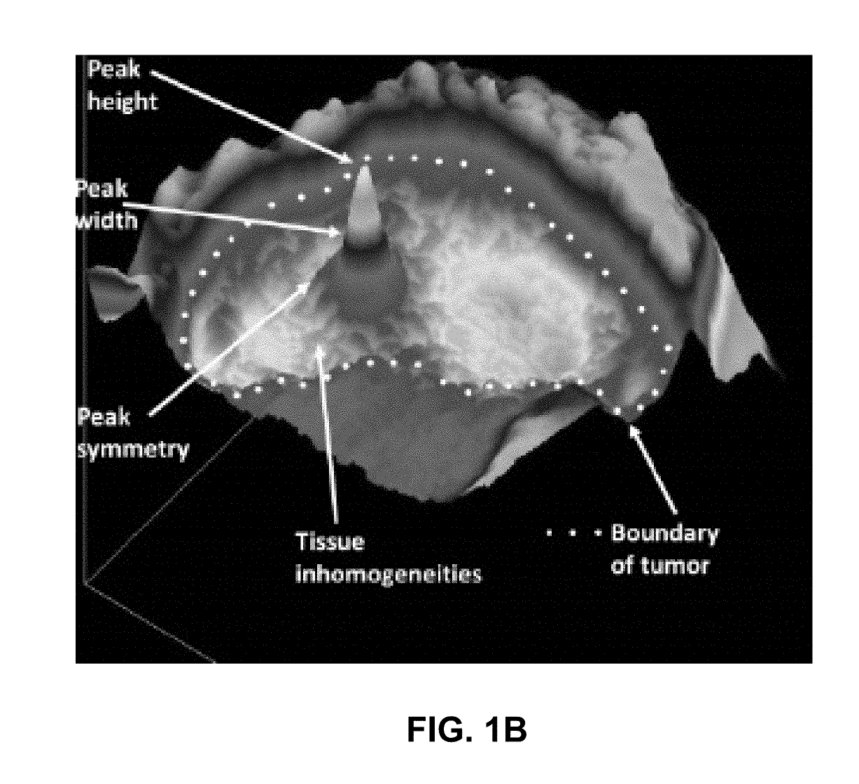

Referring again to FIG. 12, the method 1200 may further include introducing a thermal stimulation into a thermal stimulation volume within a tissue or region of interest at 1204. In various aspects, the thermal stimulation comprises an amount of heat introduced into the relatively small thermal stimulation volume positioned within the thermally imaged tissue. In one aspect, the thermal stimulation volume may be positioned within a region of interest to be thermally imaged, and may further be selected to comprise a relatively small fraction of the total area of the region of interest. By way of non-limiting example, the thermal stimulation volume may be confined to a small spot within a suspected tumor, as illustrated in FIG. 1A. In various other aspects, selection of the size of the thermal stimulation volume may be influenced by one or more additional factors including, but not limited to, the resolution of imaging device, the size or volume of tumor, the total amount of heat to be added, and any other relevant additional factor.

In various aspects, the size of the thermal stimulation volume may be influenced by at least one of a plurality of factors including, but not limited to, the detection sensitivity of the thermal imaging device, the spatial resolution of the thermal imaging device, a predicted size range of a tumor to be identified, an amount of heat to be added as the thermal stimulation, and any other relevant factor. In one aspect, the thermal stimulation may be delivered to a thermal stimulation volume no larger than the spatial resolution of the thermal imaging device, so that the thermal stimulation may function approximately as a point heat source. In another aspect, the thermal stimulation volume may be selected based on an a predicted size range of a tumor to be identified, so that the thermal stimulation volume comprises no more than about 10% of the predicted tumor volume. In an additional aspect, the thermal stimulation volume may be selected to provide sufficient volume to receive the thermal stimulation and exhibit a detectably high temperature increase without further inducing thermal damage to the tissues within thermal stimulation volume.

In various other aspects, the amount of heat to be added to the thermal stimulation volume may be influenced by at least one of a plurality of factors including, but not limited to, the detection sensitivity of the thermal imaging device, a predicted size range of a tumor to be identified, an amount of heat to be added as the thermal stimulation, and any other relevant factor. In one aspect, the amount of heat to be introduced into the thermal stimulation volume may be selected to induce a detectably high temperature increase of the thermal stimulation volume, and to diffuse from the thermal stimulation volume into surrounding tissues within the region of interest in a detectable pattern of elevated temperatures, as illustrated in FIG. 1A. In another aspect, the amount of heat to be added to the thermal stimulation volume may be limited to prevent tissue damage.

In an additional aspect, the amount of heat may be introduced into the thermal stimulation volume at a relatively high heat transfer rate to produce a thermal stimulation capable of functioning approximately as a point heat source. In some aspects, the maximum rate of heat transfer may be influenced by one or more additional factors including, but not limited to, the size or volume of tumor, the prevention of thermal damage to the tissue within the thermal stimulation volume, the shortening of data acquisition time, the desired temperature increase within thermal stimulation volume, and the desired spatial extent of the heat transfer field.

Any suitable heat source may be used to deliver the thermal stimulation without limitation. In one aspect, the thermal stimulation may be provided using a single source or a combination of sources. Non-limiting examples of suitable heat sources include electromagnetic radiation sources such as lasers or LEDs of various wavelengths, radiofrequency sources, chemiluminescence sources, and bioluminescence sources; acoustic or vibrational sources including ultrasound transducers; radioactive sources including imaging agents or therapeutic agents such as radiopharmaceuticals and other radionuclides; chemical sources including pyretic and anti-pyretic products that spontaneously generate or reduce heat or inflammation, such as some analgesics including menthol; biological sources that modulate body heat such as interleukin and interferons, and bioluminescence agents; and engineered material heat sources configured to generate or absorb heat such as thermosensitive chemical compounds, drugs, biologics, nanoparticles, radiofrequency stimulated heat sources such as dielectric heating devices that produce heat using medium frequency alternating current ranging from about 350 kHz to about 500 kHz, and high frequency stimulated heat sources; and any other suitable heat source.

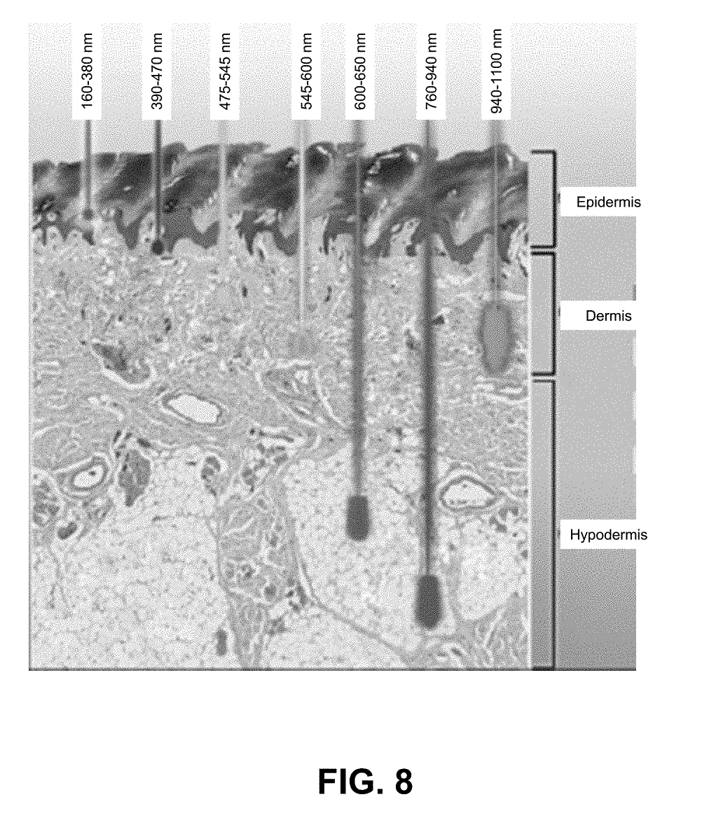

In one aspect, the thermal stimulation is provided by light sources. Without being limited to any particular theory, light penetrates tissue differently depending upon the wavelength of light, with short wavelengths penetrating a few hundred micrometers, and long wavelengths penetrating multiple millimeters, as illustrated in FIG. 8. In another aspect, thermal stimulation may be introduced into thermal stimulation volumes positioned at various depths below the skin using light sources configured to produce light at different excitation wavelengths. In some aspects, a light source producing light at a single wavelength may be used to introduce thermal stimulation at a single depth below the skin surface of the patient. In other aspects, two or more light sources, in which each light source is configured to produce light at a different wavelength, may be used to introduce thermal stimulation at two or more depths below the skin surface of the patient. In these other aspects, the two or more light sources may be operated sequentially to produce a series of discrete single-wavelength thermal stimulations, or the two or more light sources may be operated simultaneously to enable simultaneous multispectral wavelength thermal stimulation that includes excitation light of two or more different wavelengths.

In one aspect, computational modeling may be used to estimate the effects of laser wavelength, power, spot size, and illumination duration on LSTI heat diffusion within tissue. The results of this computational modeling aid in understanding the impact of each variable in the LSTI process, and may further be used to develop classification algorithms for identifying disease and precancerous and/or cancerous lesions.

In another aspect, the thermal stimulation is provided by acoustic sources configured to direct ultrasound energy into the thermal stimulation volume. Without being limited to any particular theory, ultrasound energy typically penetrates biological tissues deeper below the skin surface as compared to the penetration depths of light energy. In this other aspect, ultrasound waves, such as that utilized in high intensity focused ultrasound, may be used to generate heat deep within tissues, overcoming optical penetration depth limitations.

In other additional aspects, at least a portion of the thermal stimulation may be provided or enhanced by exogenous compounds administered to the patient in combination with one or more additional sources including, but not limited to, light sources and acoustic sources. Any exogenous compound capable of absorbing energy introduced from an external source such as a laser or an ultrasound transducer, and converting the absorbed energy into heat, may be selected for use as an exogenous compound without limitation. Non-limiting examples of suitable exogenous compounds include photoluminescent contrast agents, photothermal contrast agents, photoacoustic contrast agents, and ultrasound contrast agents. In various other aspects, the exogenous compounds may be functionalized and/or linked to additional moieties configured to target particular cell types including, but not limited to, tumor cells.

In various additional aspects, the exogenous compounds may include moieties configured to generate heat in response to externally applied light or acoustic energy, as well as therapeutic moieties configured to enable treatments of disorders including, but not limited to, a cancer. In some aspects, the therapeutic moieties may be configured to be inactive until exposed to activation stimuli including, but not limited to, light, heat, acoustic, and/or electrical energy. Non-limiting examples of suitable exogenous compounds are described in U.S. Pat. No. 8,053,415, the contents of which are incorporated by reference herein in its entirety.

In other additional aspects, heat-generating contrast agents may be used to enable theranostic treatment methods in addition to enabling light-stimulated thermal imaging methods as described herein. Non-limiting examples of agents suitable for enabling theranostic treatment methods in these other additional aspects include LS301, 5-ALA, Technetium 99, and FDG. These agents would allow for localization of the tumor using LSTI while applying a stimulus to release the therapeutic load.

By way of non-limiting example, an exogenous compound that includes a contrast moiety functionalized to target a tumor cell and a treatment moiety configured to enable a treatment of the targeted tumor cell may be administered to a patient. In this non-limiting example, upon administration to the patient, the exogenous compound may bind to a tumor cell. The exogenous compound may produce a thermal stimulation in response to illumination by a laser source, resulting in the detection of the cancer cell/tumor tissue according to the laser-stimulated thermal imaging methods described herein. Optionally, the laser light may additionally act as a stimulus to activate the treatment moiety to enable a treatment of the tumor cell, or the treatment moiety may be activated using a different stimulus.

In various additional aspects, the thermal stimulation may be provided to a plurality of thermal stimulation volumes to enable wide field laser-stimulated thermal imaging. In these additional aspects, thermal stimulations are introduced at a spatial array of thermal stimulation volumes to enable laser-stimulated thermal imaging over a relatively large area of tissue. In some aspects, the plurality of thermal stimulations may be introduced simultaneously over a relatively large tissue area containing the array of thermal stimulation volumes. By way of non-limiting examples, the thermal stimulations may be provided in the form of a 2D array of laser spots configured to irradiate tissue simultaneously to create a grid of thermal profiles over an area. In other aspects, a single laser source may be used to create a grid of thermal profiles over an area by produce a sequence of thermal stimulations while translating the single laser source over a scanning pattern. Non-limiting examples of suitable scanning patterns include line scans, raster scans, concentric circular scans, spiral scans, and any other suitable scanning pattern.

In various aspects, the method 1200 may further include obtaining at least one additional thermal image of the tissue at 1206 after introducing the thermal stimulation at 1204. In various aspects, the at least one additional thermal image may be obtained at 1206 using the same thermal imaging device used to obtain the baseline thermal image of the tissue at 1202 as described above. In one aspect, a single additional thermal image may be obtained at 1206 after sufficient time has passed to form a detectable thermal pattern in the tissues surrounding the thermal stimulation volume after the introduction of the thermal stimulation at 1204. In various other aspects, two or more additional thermal images may be obtained at 1206 at two or more time intervals after the introduction of the thermal stimulus at 1204. In these various other aspects, the two or more additional thermal images obtained at 1206 may enable the assessment of additional thermal characteristics of the tissue including, but not limited to, a rate of change of a spatial temperature distribution as a function of time after introduction of the thermal stimulation.

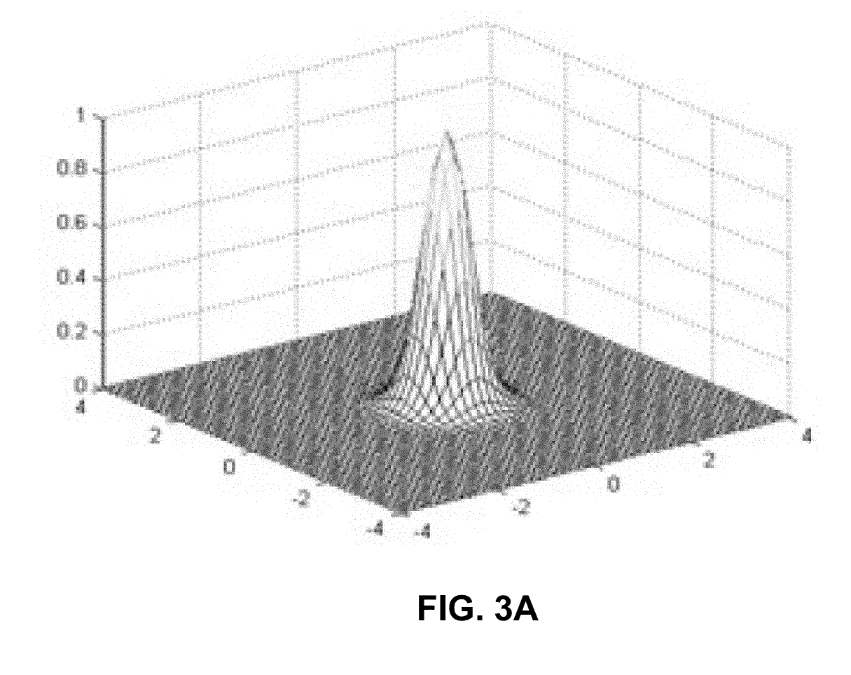

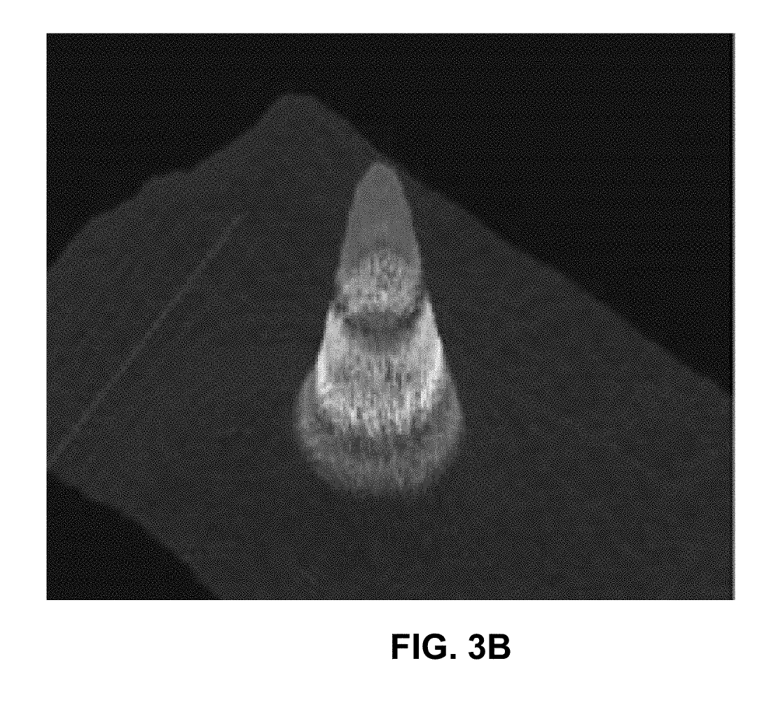

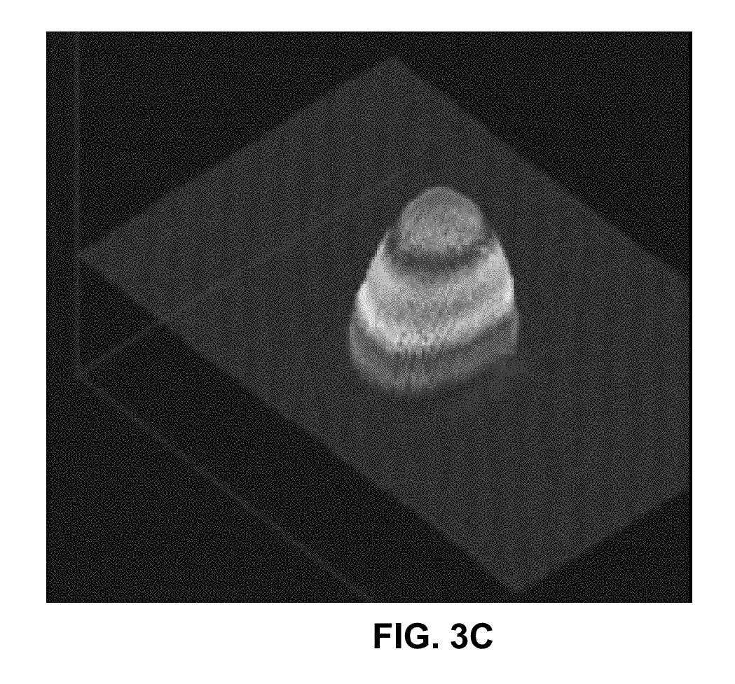

Referring again to FIG. 12, the method 1200 may further include creating a thermal diffusion profile at 1208 based on at least one of the thermal images obtained at 1202 and 1206. In one aspect, each thermal diffusion profile is a spatial map of temperatures that includes an array of pixels associated with the at least one thermal image and a temperature corresponding to each pixel within a region of interest of the at least one thermal image. The thermal diffusion profile may be displayed using any suitable format without limitation. In one aspect, the thermal diffusion profile may be provided to the practitioner in any suitable format including, but not limited to, a numerical array, a color-mapped image, a color-mapped contour image, a wire-frame contour, and any other suitable format. By way of non-limiting example, FIG. 1A is a thermal diffusion profile provided in the form of a color-mapped image, in which the colors within the image encode the temperature corresponding to each pixel location within the image. By way of another non-limiting example, FIG. 3A is a simulated thermal diffusion profile provided in the form of a 3D contour, in which the z-value (i.e. height) at each pixel location encodes the temperature corresponding to each pixel within the thermal image. FIG. 1B is a thermal diffusion profile provided in the form of a color-mapped contour image by way of an additional non-limiting examples.

In one aspect, the thermal diffusion profile may be created at 1208 by pixel-wise subtraction of the temperatures of the thermal image obtained prior to the thermal stimulation (i.e. the thermal image obtained at 1202) from the temperatures of the thermal image obtained after thermal stimulation (i.e. the thermal image obtained at 1206). In this aspect, the thermal diffusion profile includes a map of temperature changes induced within the tissue by the thermal stimulation introduced at 1204. In another aspect, the thermal diffusion profile may be created at 1208 directly from the thermal image obtained after thermal stimulation (i.e. the thermal image obtained at 1206).

In various other aspects, if two or more images of the thermally stimulated tissues are obtained at 1206, the spatial temperature plots created at 1208 may be a series of two or more plots. Each plot within the series may include the temperature at each time after thermal stimulation obtained directly from each thermal image obtained at 1206, a change in temperature relative to the tissue temperatures prior to thermal stimulation, or an incremental change in temperature relative to a preceding thermal image obtained at 1206.

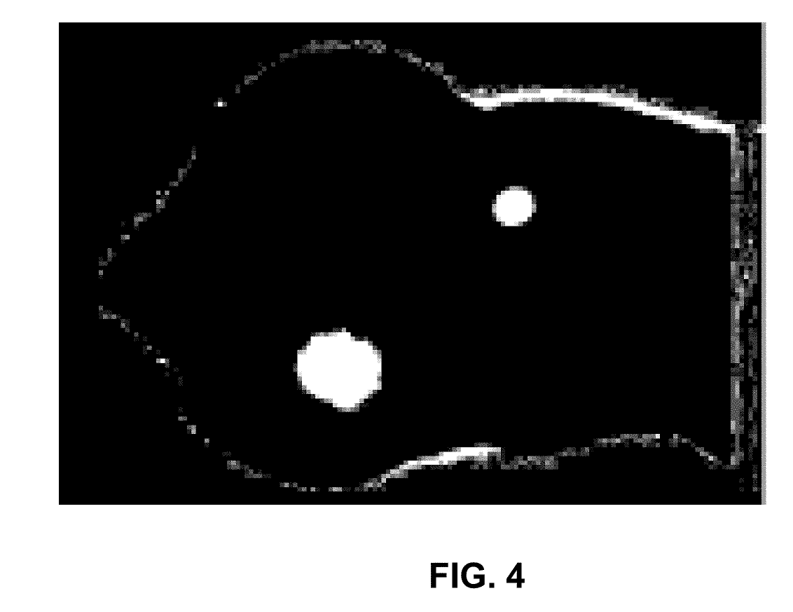

Without being limited to any particular theory, the spatial temperature plots obtained at 1208 may include at least one observable feature indicative of a tissue type including, but not limited to, healthy tissue and tumor tissue. Non-limiting examples of observable features of spatial temperature plots that may be indicative of a tissue type include peak height, profile width, profile symmetry, profile boundary shape, and inhomogeneities within the profile boundary. By way of non-limiting example, FIG. 4 is an image showing spatial temperature plots that include heating profiles obtained from tumor tissue (lower left spot) and normal tissue (upper right spot), in which the profile boundary of the tumor tissue is relatively irregular compared to the corresponding profile boundary of the normal tissue.

Referring again to FIG. 12, the method 1200 may further include calculating one or more thermal diffusion parameters at 1210 based on an analysis of the spatial temperature plot created at 1208. Any suitable thermal diffusion parameter may be calculated at 1210 without limitation, including parameters related to temperature values, changes in temperature values with respect to time, gradients of temperature change as a function of position relative to the thermal stimulation volume, and rates of change of temperature values with respect to time or spatial position.

In various aspects, the one or more thermal diffusion parameters may include any quantifiable aspect of the spatial temperature plot without limitation. In one aspect, the one or more thermal diffusion parameters may enable the classification of a tissue type and/or a disorder including, but not limited to, cancer or tumor tissue. Non-limiting examples of suitable thermal diffusion parameters include: spatial thermal diffusion profiles of the material, with or without stimulation; spatial thermal hot-spot profile after stimulation of the material including Gaussian profiling; and temporal thermal profile of the material, before or after stimulation.

The one or more thermal diffusion parameters may be calculated using any suitable algorithm or method of analyzing spatial and temporal data variations without limitation. In one aspect, the algorithm or method of analyzing spatial and temporal data variations may include calculating one or more summary parameters for a temperature profile including, but not limited to, maximum temperature, minimum temperature, mean temperature, median temperature, temperature range, standard deviation of temperature, and any other suitable summary parameter without limitation. In another aspect, the algorithm or method of analyzing spatial and temporal data variations may include assessing a shape or spatial distribution-related parameter of the thermal diffusion contour including, but not limited to, radial symmetry of the contour profile, distribution of temperatures within a thermal diffusion contour with respect to one or more statistical distributions such as a Gaussian distribution, homogeneity of the temperature distribution within the thermal contour, and any other shape or spatial distribution-related parameter without limitation. In an additional aspect, the algorithm or method of analyzing spatial and temporal data variations within a thermal diffusion contour may include assessing one or more rates or gradients including, but not limited to, rates of temperature increase over time at different spatial positions within a contour, temperature gradients within a thermal profile at one time or as a function of time, and any other suitable rate or gradient without limitation. In another additional aspect, the algorithm or method of analyzing spatial and temporal data variations within a thermal diffusion contour may include, but is not limited to, spectral or other frequency-based analysis including spatial and/or temporal Fourier analysis.

Referring again to FIG. 12, the method 1200 may further include classifying at 1212 a tissue within the region of interest based on the values of the one or more thermal diffusion parameters calculated at 1210 according to one or more classification rules. In various aspects, the classification rules may be empirically-derived by comparing the values of one or more thermal diffusion parameters obtained from a plurality of control (normal/healthy) thermal images to the corresponding one or more thermal diffusion parameters obtained from a plurality of patient thermal images. Non-limiting examples of tissue classifications that may be obtained at 1212 include: normal versus diseased tissues, including tissue biopsy for disease diagnosis and/or staging; tissues with normal physiology versus pathophysiology; functionally active tissues associated with brain function and other biological processes; tissue segmentation to identify local distribution of vascular, bone, cardiac, nerve, and/or other tissues. In various aspects, the classification rule may include an assessment of a single thermal diffusion parameter or a combination of two or more thermal diffusion parameters. By way of non-limiting example, one classification rule may be to classify the tissue as healthy tissue if the temperature distribution within the thermal diffusion profile is Gaussian and classifying the tissue as tumor tissue if the temperature distribution within the thermal diffusion profile is non-Gaussian. By way of another example, another classification rule may be to classify the tissue as healthy tissue if the contour of the thermal diffusion profile is radially symmetrical and classifying the tissue as tumor tissue if the contour of the thermal diffusion profile is asymmetrical.

In various aspect, the LSTI devices, systems, and methods may enable a number of medical applications including, but not limited to: biopsy of a tissue for disease diagnosis or staging; identifying characteristics and volume of a tissue of interest; monitoring of a treatment response to a disease; predicting efficacy of treatment in a patient to enable a personalized treatment protocol; digital pathology; vein mapping, including identification of thromboses; detection of hemorrhage; detection of neuropathy and other neurological diseases; monitoring of cardiovascular functions and processes. In various other aspects, the LSTI devices, systems, and methods may be incorporated into other medical devices and systems including, but not limited to, surgical navigation systems, treatment administration systems, and any other compatible medical devices and/or systems. By way of non-limiting example, the LSTI devices, systems, and methods may be incorporated into a surgical navigation system to facilitate in navigating surgical procedures, as well as identifying and/or monitoring patient tissues during surgery to identify tumor borders during breast tumor resection or to monitor for complications such as of vein blockage or hemorrhage.

In other additional aspects, the LSTI devices, systems, and methods may provide enhanced visualization useful for a variety of medical research initiative including but not limited to: designing and screening compounds such as drugs; developing appropriate animal models for testing disease treatment and drugs; incorporating immune response as a disease treatment.

In one aspect, the LSTI devices, systems, and methods may be used for endoscopic imaging. In this aspect, the LSTI system may be provided in the form of an endoscopic system for use in the GI tract or any other suitable endoscopic application. Depending upon the application, the endoscopic LSTI system may incorporate a laser or an ultrasound transducer to obtain stimulated thermal images. In various aspects, the endoscopic LSTI system may be suitable for visualizing a variety of disorders including, but not limited to, colon cancer, gastric cancer, inflammatory bowel disease, oral cancer, Barrett's esophagus, esophageal cancer, pancreatic cancer, liver cancer, gallbladder cancer, and anal cancer.

By way of non-limiting example, an endoscopic LSTI system may be used for cervical cancer screening. Without being limited to any particular theory, cervical cancer screening paired with treatment is extremely effective at preventing advanced stage cervical cancer. However, in low resource settings that typically have relatively large cervical cancer burdens, cervical cancer screening may be challenging due to limited training and equipment. In one aspect, an endoscopic LSTI system may be integrated with a treatment system including, but not limited to, a cryotherapy system. In this other aspect, the integrated endoscopic LSTI/cryogenic treatment system may be used to implement a see-and-treat method and may facilitate the expansion of access to accurate cervical cancer screening and treatment to patients in low resource settings and remote areas.

In another aspect, the LSTI systems and devices may be integrated into a surgical guidance system. In this other aspect, the thermal imaging device of the LSTI system may be operatively coupled to the surgical guidance system such that the digitally enhanced thermal images and/or thermal diffusion profile data may be registered with other imaging modalities of the surgical guidance system and displayed to a user/practitioner to provide surgical guidance information. In one aspect, the surgical guidance system with integrated LSTI capability may be used in a variety of surgical interventions including, but not limited to, breast tumor resection surgery.

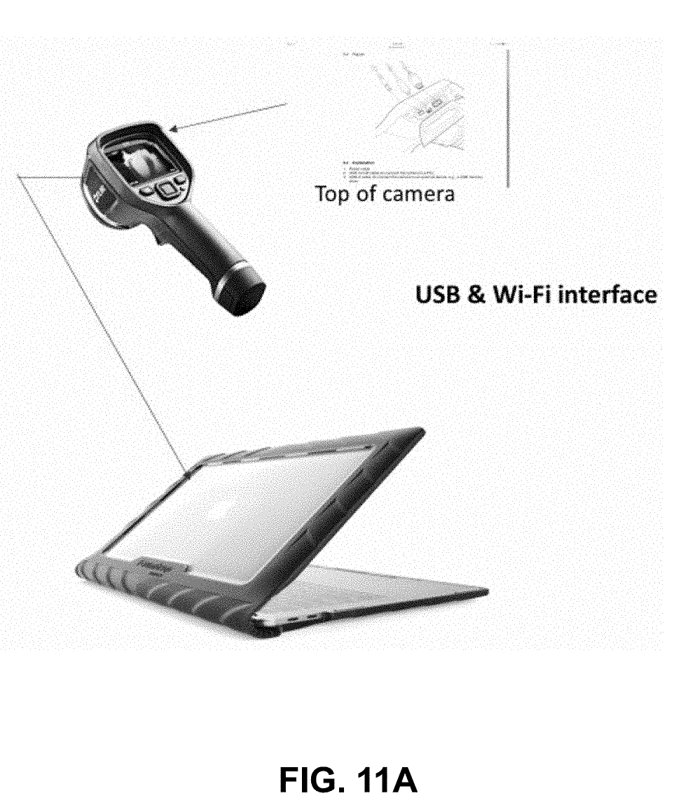

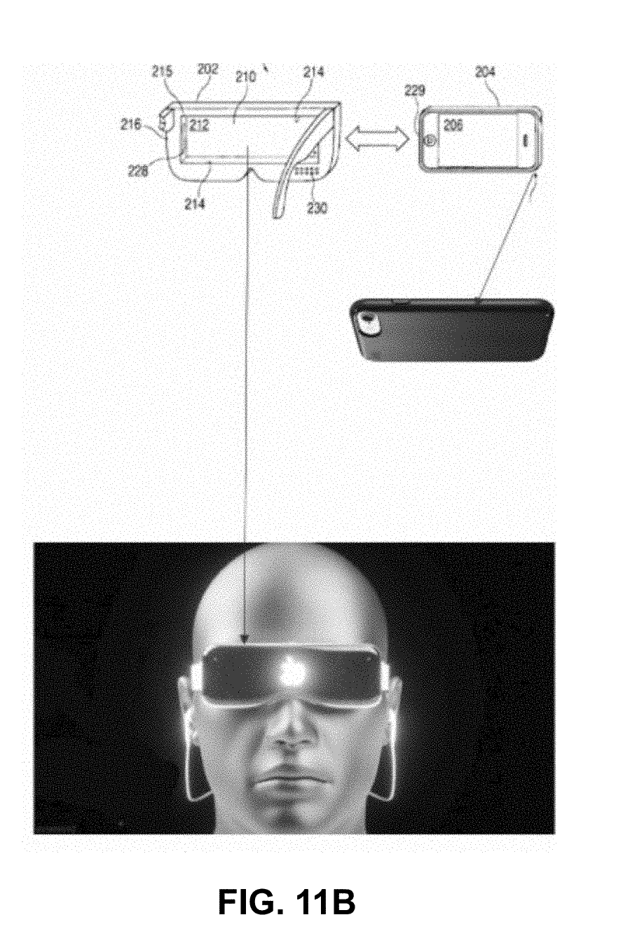

In various aspects, the LSTI systems and devices may be provided in a variety of formats including, but not limited to, goggles, handheld systems, portable bedside imaging systems, and any other suitable format without limitation. In one aspect, the LSTI systems and devices may be provided in the form of thermal imaging goggles. In this aspect, the thermal imaging goggles may be optimized for use at the point of care and for intra-operative surgical guidance. In an additional aspect, the thermal imaging goggles may be combined with fluorescence to multiplex information and enhance detection accuracy in a multimodal fluorescence and thermal imaging goggle system. FIG. 11 is an illustration of an LSTI system that includes thermal imaging goggles in one aspect.

In various other aspects, the LSTI systems and devices may make use of commercially available handheld thermal imaging cameras, as illustrated in FIG. 9. These handheld thermal imaging cameras are portable, typically equipped with batteries lasting over 4 hours, and are configured to easily transmit images to a processor of a computing device. In one aspect, an LSTI system with a handheld thermal imaging camera may be configured to enable real-time data acquisition and analysis. In another aspect, an LSTI system may be a standalone enhanced thermal imaging system that includes a laser, thermal camera, and processor.

In various other aspects, the LSTI system may be a wide-field LSTI system suitable for use in breast cancer screening in point-of-care settings. In one aspect, the wide-field LSTI system may include an ultrasound transducer to enable ultrasound stimulated thermal imaging suitable for breast cancer screening. The wide-field LSTI system may be portable, battery powered, hand-held, and configured to produce test results within seconds so that patients could be immediately triaged if additional medical care and expertise is indicated.

In another aspect, the LSTI system may be configured to enable whole-body skin cancer and skin lesion screening. In this aspect, the thermal imaging device may be integrated into a whole-body chamber configured to receive a whole body of patient, similar to millimeter wave detection scanners deployed for security screenings at secured locations such as airports and court houses. In this other aspect, the whole-body chamber of the LSTI system may be configured to perform full body screening for skin lesions and any other suitable dermatological disorder without limitation.

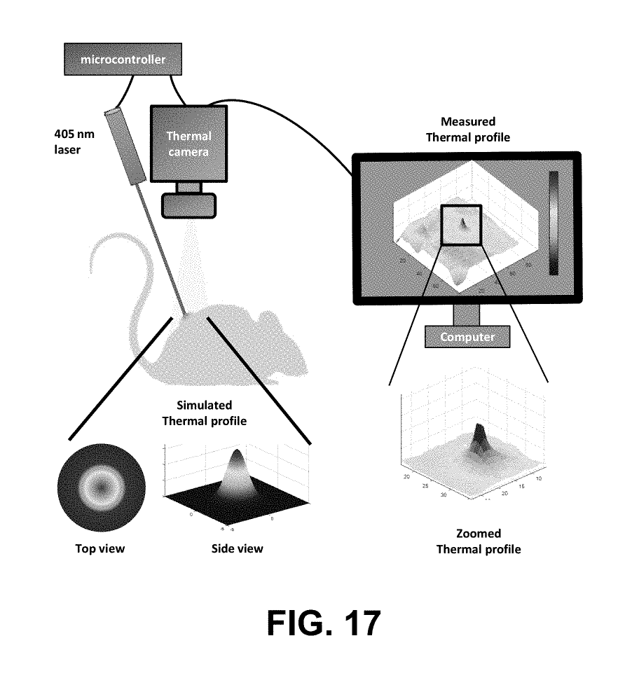

In one aspect, the LSTI system is an automated LSTI system in which laser stimulation and thermal video imaging and processing are enabled with the click of a button. FIG. 17 is a schematic illustration showing the elements of an automated LSTI system. In some aspects, an automated LSTI system interrogates suspicious spots in less than 20 seconds. Non-limiting characteristics of an automated LSTI system include being non-contact, label-free, low cost, portable, performing real time analysis/processing, and/or utilizing non-ionizing radiation.

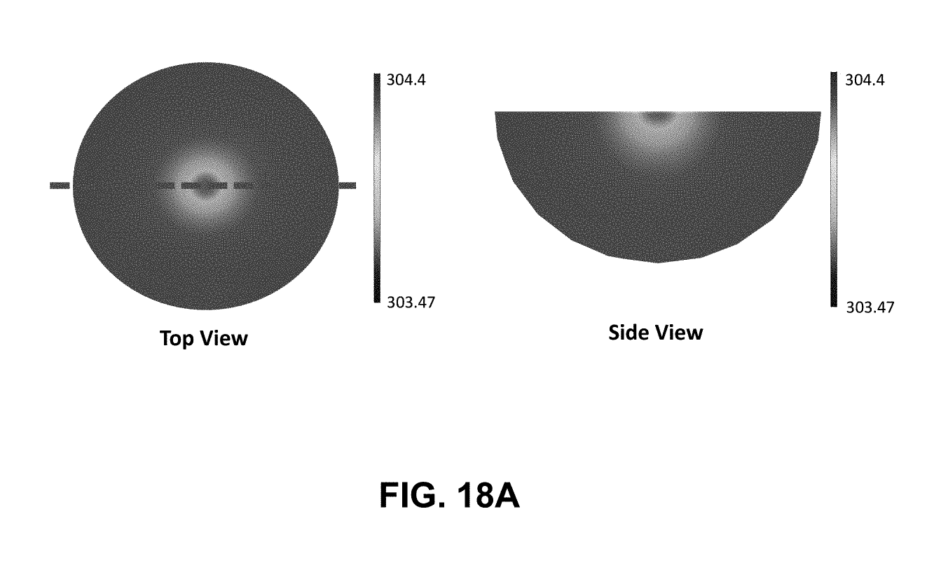

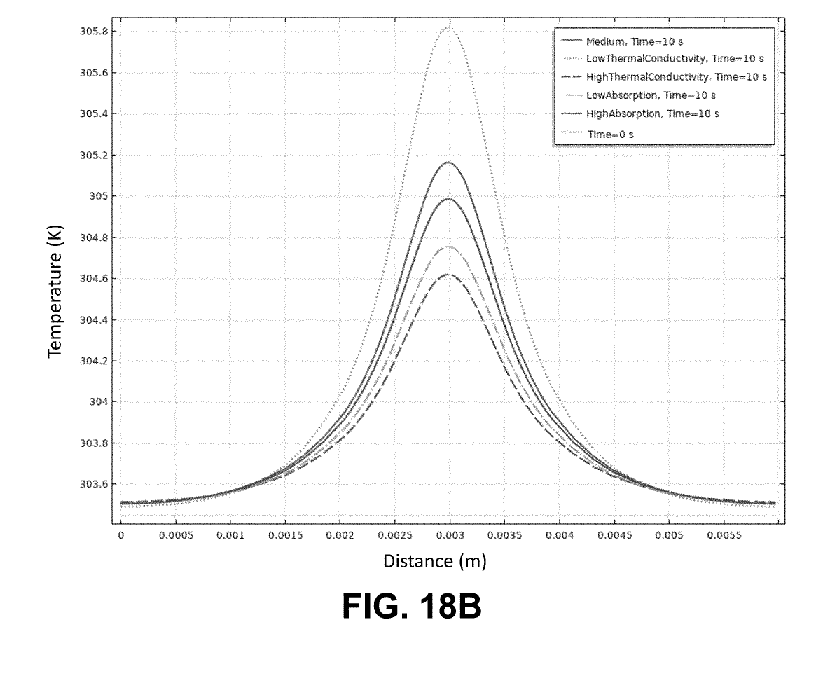

In various aspects, computational simulation demonstrates sensitivity to differences in tissue thermal conductivity and optical absorption-biochemical specific parameters. FIG. 18A illustrates a top view and side view model based on Penne's Bioheat Equation while FIG. 18B illustrates a plot of surface temperature cross-section as shown in FIG. 18A. Various aspects of computational simulation are also shown in the Examples disclosed herein.

In various aspects, the digitally enhanced thermal imaging methods and LSTI methods described above may be deployed on at least one computing device. In various other aspects, the digitally enhanced thermal imaging methods and LSTI methods described above may be deployed on one or more computing devices that are operatively coupled to one or more devices including, but not limited to, thermal imaging devices, as well as therapy delivery devices including, but not limited to, radiation therapy delivery devices and cryotherapy devices.

In one aspect, an LSTI computer system includes at least one computing device and at least one database. In one aspect, the database and computing device are components of a server system. The server system may be a server, a network of multiple computer devices, a virtual computing device, or the like. In some aspects, the computing device is communicably coupled to a network which allows communication with a plurality of computing devices. For example, the computing device may be able to communicate with a plurality of user computing devices, therapy control devices, thermal imaging devices, and/or other types of computer devices or sensors.

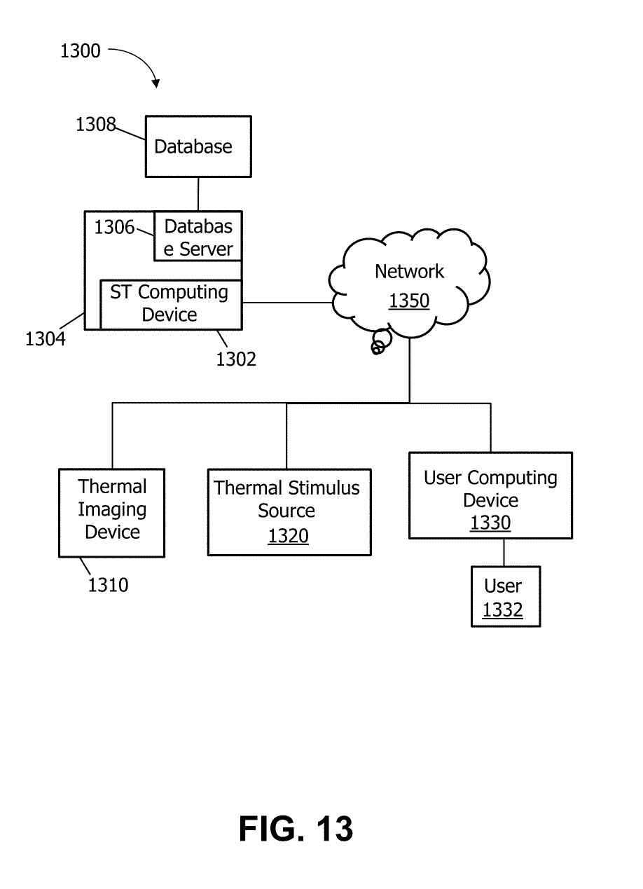

FIG. 13 illustrates an LSTI computer system 1300 implemented in a medical setting. In the one aspect, the LSTI computer system includes a computing device 1302. The computing device 1302 is part of a server system 1304, which includes a database server 1306. The computing device 1302 is in communication with the database 1308 through the database server 1306. The computing device 1302 is communicably coupled to a thermal imaging device 1310, a thermal stimulus source 1320, and a user computing device 1330 through the network 1350. Network 1350 may be any network that allows local area or wide area communication between devices. For example, network 1350 may allow communicative coupling to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. User computer device 1330 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

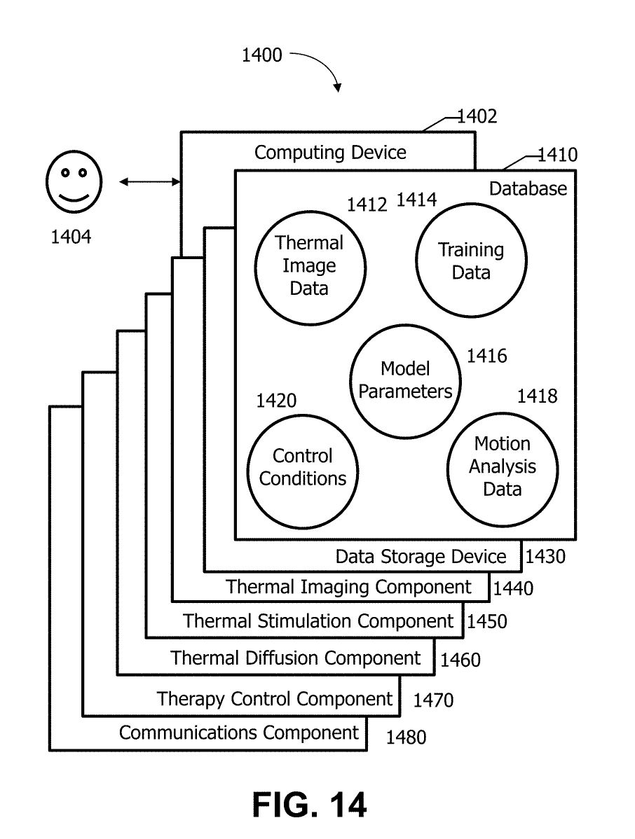

FIG. 14 depicts a component configuration 1400 of computing device 1402, which includes database 1420 along with other related computing components. In some aspects, computing device 1402 is similar to computing device 1302 (shown in FIG. 13). User 1404 may access components of computing device 1402. In some aspects, database 1410 is similar to database 1308 (shown in FIG. 13).

In the example aspect, database 1410 includes thermal image data 1412, spatial temperature plot data 1414, thermal diffusion parameters 1416, tissue classification data 1418, and device control parameters 1420. Thermal image data 1412 may include, but is not limited to, thermal images and thermally stimulated thermal images received from a thermal imaging device as described above. Spatial temperature plot data 1414 may include data defining one or more spatial temperature plots as defined above. Thermal diffusion parameters 1416 may include one or more of the thermal diffusion parameters determined from the spatial temperature plots as described above. Tissue classification data 1418 may include data defining the classifications of tissues based on the thermal diffusion parameters as described above. Device control parameters 1420 a plot plurality of parameters defining the control of one or more additional devices as described above including, but not limited to, a thermal imaging device, a thermal stimulation source, a therapy administration device, and any other device of the system.

The computing device 1402 also includes a number of components which perform specific tasks. As illustrated in FIG. 14, the computing device 1402 includes data storage device 1430, thermal imaging component 1440, thermal stimulation component 1450, thermal diffusion component 1460, therapy control component 1470, and communications component 1480. Data storage device 1430 is configured to store data received or generated by computing device 1402, such as any of the data stored in database 1410 or any outputs of processes implemented by any component of computing device 1402. For example, in some aspects data storage device 1430 stores thermal diffusion plots and parameters. Thermal imaging component 1440 is configured to operate a thermal imaging device to obtain thermal images as described above. Thermal stimulation component 1450 is configured to operate a thermal stimulation source in coordination with the thermal imaging device to thermally stimulate a tissue prior to obtaining a thermal image as described above.

Referring again to FIG. 14, thermal diffusion component 1460 is configured to create a spatial temperature plots based on the one or more thermal images as described above. In another aspect, thermal diffusion component 1460 is further configured to determine at least one thermal diffusion parameter based on the spatial temperature plots. In an additional aspect, the thermal diffusion component 1460 is further configured to classify tissue within the thermal images based on the at least one thermal diffusion parameter. Therapy control component 1470 is configured to operate a therapy administration device based on information obtained using the LSTI system. In some aspects, therapy control component 1470 generates instructions for increasing, decreasing, or changing a type of therapy output by a therapy device based on tissue type, thermal diffusion parameter, or any quantity or parameter generated by the thermal diffusion component 1460. Communications component 1480 is configured to enable communications between computing device 1402 and other computing devices (e.g. user computing device 1330, thermal stimulus source 1320, and thermal imaging device 1310, all shown in FIG. 13) over a network, such as network 1350 (shown in FIG. 13), or a plurality of network connections using predefined network protocols such as TCP/IP (Transmission Control Protocol/Internet Protocol).

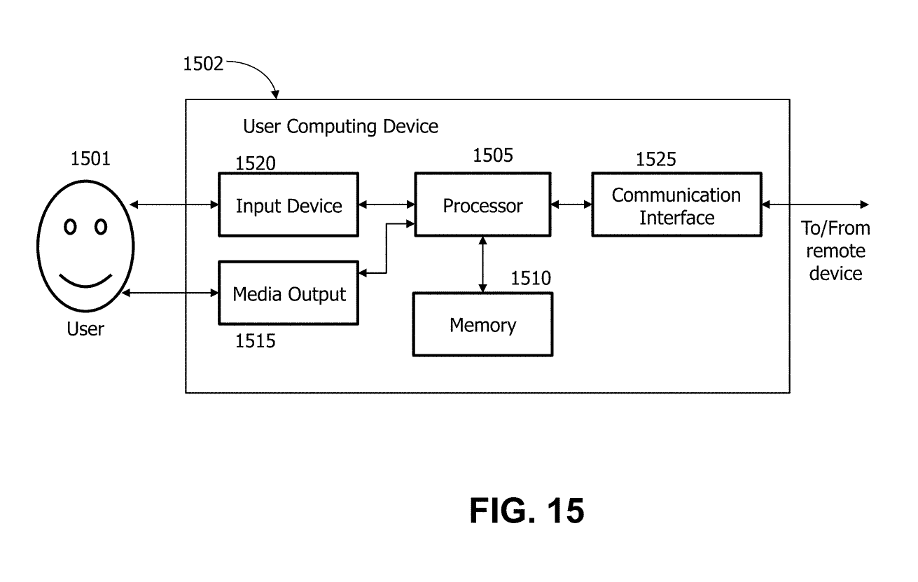

FIG. 15 depicts a configuration of a remote or user computing device 1502, such as user computing device 1330 (shown in FIG. 13). Computing device 1502 may include a processor 1505 for executing instructions. In some aspects, executable instructions may be stored in a memory area 1510. Processor 1505 may include one or more processing units (e.g., in a multi-core configuration). Memory area 1510 may be any device allowing information such as executable instructions and/or other data to be stored and retrieved. Memory area 1510 may include one or more computer-readable media.

Computing device 1502 may also include at least one media output component 1515 for presenting information to a user 1501. Media output component 1515 may be any component capable of conveying information to user 1501. In some aspects, media output component 1515 may include an output adapter, such as a video adapter and/or an audio adapter. An output adapter may be operatively coupled to processor 1505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 1515 may be configured to present an interactive user interface (e.g., a web browser or client application) to user 1501.

In some aspects, computing device 1502 may include an input device 1520 for receiving input from user 1501. Input device 1520 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 1515 and input device 1520.

Computing device 1502 may also include a communication interface 1525, which may be communicatively coupleable to a remote device. Communication interface 1525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 1510 are, for example, computer-readable instructions for providing a user interface to user 1501 via media output component 1515 and, optionally, receiving and processing input from input device 1520. A user interface may include, among other possibilities, a web browser and client application. Web browsers enable users 1501 to display and interact with media and other information typically embedded on a web page or a website from a web server. A client application allows users 1501 to interact with a server application associated with, for example, a vendor or business.

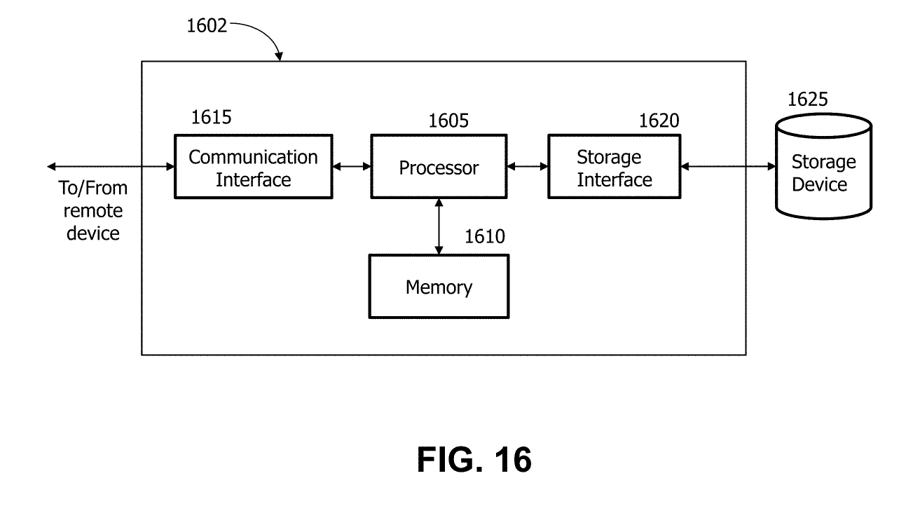

FIG. 16 illustrates an example configuration of a server system 1602. Server system 1602 may include, but is not limited to, database server 1306 and computing device 1302 (both shown in FIG. 13). In some aspects, server system 1602 is similar to server system 1304 (shown in FIG. 13). Server system 1602 may include a processor 1605 for executing instructions. Instructions may be stored in a memory area 1610, for example. Processor 1605 may include one or more processing units (e.g., in a multi-core configuration).

Processor 1605 may be operatively coupled to a communication interface 1615 such that server system 1602 may be capable of communicating with a remote device such as user computing device 1330 (shown in FIG. 13) or another server system 1602. For example, communication interface 1615 may receive requests from user computing device 1330 via a network 1350 (shown in FIG. 13).

Processor 1605 may also be operatively coupled to a storage device 1625. Storage device 1625 may be any computer-operated hardware suitable for storing and/or retrieving data. In some aspects, storage device 1625 may be integrated in server system 1602. For example, server system 1602 may include one or more hard disk drives as storage device 1625. In other aspects, storage device 1625 may be external to server system 1602 and may be accessed by a plurality of server systems 1602. For example, storage device 1625 may include multiple storage units such as hard disks or solid state disks in a redundant array of inexpensive disks (RAID) configuration. Storage device 1625 may include a storage area network (SAN) and/or a network attached storage (NAS) system.

In some aspects, processor 1605 may be operatively coupled to storage device 1625 via a storage interface 1620. Storage interface 1620 may be any component capable of providing processor 1605 with access to storage device 1625. Storage interface 1620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 1605 with access to storage device 1625.

Memory areas 1510 (shown in FIG. 15) and 1610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one aspect, a computer program is provided, and the program is embodied on a computer readable medium. In one aspect, the system is executed on a single computer system, without requiring a connection to a sever computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

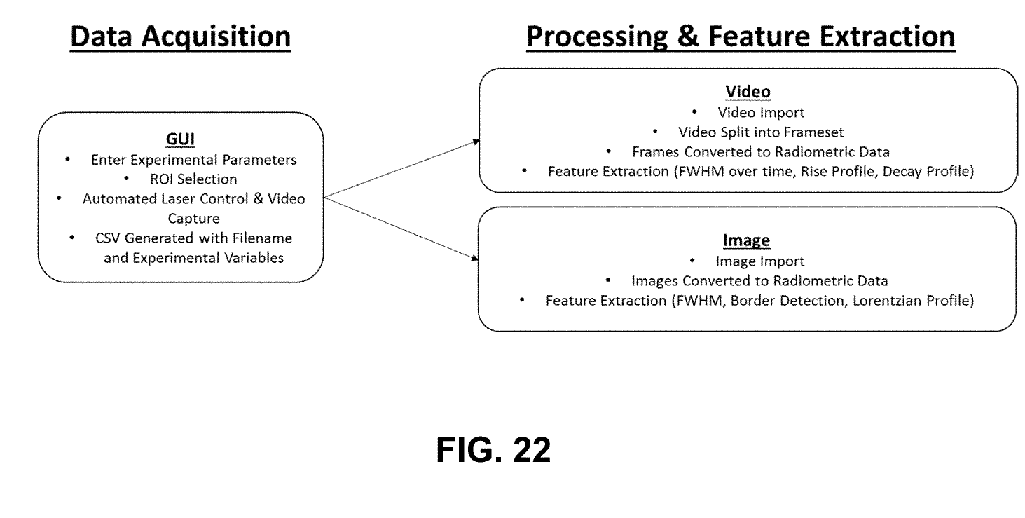

In various aspects, the disclosed systems and methods may be used to enable the production and analysis of images using laser-stimulated thermal imaging (LSTI) as disclosed above. FIG. 22 is a schematic diagram illustrating various features of image production and analysis for use in a clinical setting in various aspects. The acquisition of the laser-stimulated thermal imaging (LSTI) images may be controlled by a user by entering data via a GUI displayed on a computing device as described above. In various aspects, the user-entered data may encode information such as experimental parameters, specification of a region of interest (ROI) to be subjected to LSTI, parameters used to operate the laser and video camera in a coordinated manner, and to generate and store a data file, such as a CSV document, containing the experimental variables and any other information relevant to the LSTI images without limitation.

Referring again to FIG. 22, the LSTI images are obtained in the form of a video clip or as a series of single images in various aspects. In one aspect, a video clip obtained using LSTI is imported, split into a frameset containing a plurality of individual frames, and each frame is converted into radiometric data. In another aspect, each image of a series of images is imported and converted into radiometric data. In yet another aspect, the radiometric data obtained from the imported video or imported series of individual images as described above are subjected to feature extraction to automatically identify any one or more of at least several features relevant to the analysis of the LSTI images including, but not limited to, FWHM, detected borders, and Lorentzian profiles.

### EXAMPLES

The following examples illustrate various aspects of the disclosure.

### Example 1: Digitally Enhanced Thermal Imaging

To validate the digitally enhanced thermal imaging methods described above, the following experiments were conducted.

A subcutaneous mouse model of breast cancer was used in these experiments. Six-week old balb/c mice were implanted with 10⁶ 4T1 murine breast cancer cells in the right dorsal flank. These mice were subjected to thermal imaging using the methods as described above 7-10 days after tumor implantation, when the implanted tumors were at least 1 cm in size and became visible and palpable. During the thermal imaging experiments the mice were anesthetized using isoflurane.

Twenty two balb/c mice with subcutaneous breast tumors were anaesthetized and imaged using a thermal camera (FLIR E60, resolution 320*240 pixels, sensitivity <0.05° C., temperature range −20° C. to 650° C.). The fur from the mice was shaved around the tumor to allow for thermal measurement. The thermal camera was fixed at 20 cm above the mouse and videos and snapshots were obtained. The thermal images were analyzed using MATLAB to identify distinct thermal signatures of the tumors. A temperature distribution profile was created using MATLAB (Mathworks) to visualize the spatial distribution of temperature and to identify a difference in temperature of the tumor tissue and other tissues surrounding the tumor. Sensitivity and specificity of tumor detection were calculated using prior knowledge of tumor and non-tumor tissue location.

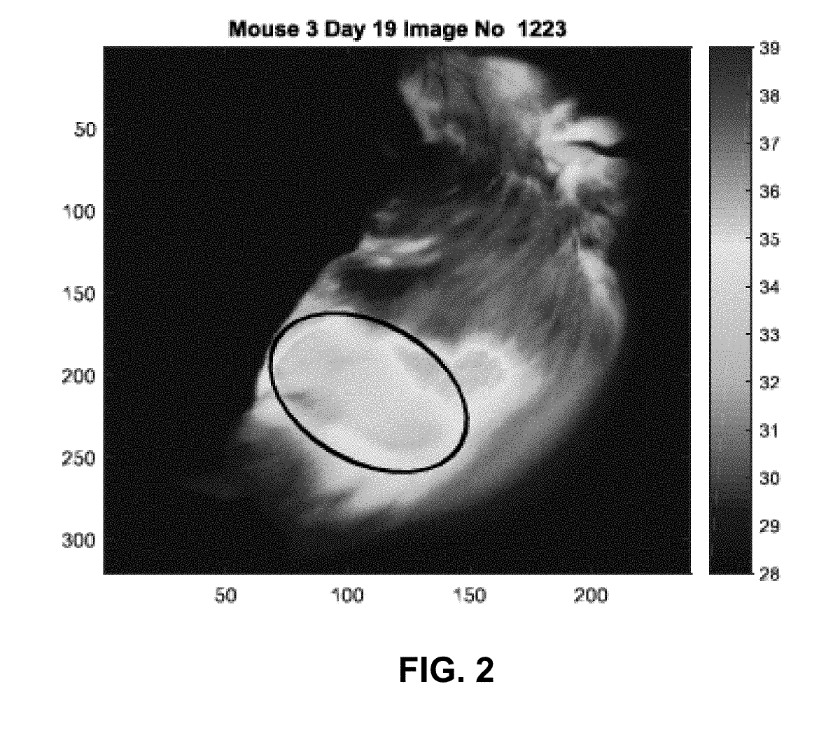

FIG. 2 is a representative thermal image obtained in this experiment as described above, with the previously-obtained margin of a tumor demarcated by a solid ellipse overlaid over the thermal image. Typically, tissue heterogeneity within portions of tumors resulted in a differential response to thermal stimulation. Further, human cancer generally appears hotter than surrounding tissue in unstimulated thermal images, but many animal models of cancer exhibit lower heat than surrounding uninvolved tissue. In this experiment, the tumors in some cases did not appear to have an obvious temperature difference compared with surrounding tissue and contralateral tissue in the thermal images.

Using conventional thermography, we found that the sensitivity (47%), specificity 57%), positive predictive value (61%), negative predictive value (43%), and accuracy (51%) were all relatively low, confirming the challenges in using this technique to make clinical decisions.

### Example 2: Laser-Stimulated Digitally Enhanced Thermal Imaging

To validate the laser-stimulated digitally enhanced thermal imaging methods described above, the following experiments were conducted.

Digitally enhanced thermal imaging and laser-stimulated digitally enhanced thermal imaging were performed as described below. Although it was previously observed that small animal models of tumors generally exhibit lower temperatures than human cancer, no cause of this difference has been definitively identified. Regardless of the thermal status of tumors relative to surrounding tissues in the absence of thermal stimulation, by thermally stimulating tumors, using either heating or cooling, detectable response characteristics associated with tumor heterogeneity result, as illustrated in FIG. 1. As a result, response to thermal stimulation may serve as a universal diagnostic factor for all types of cancer.

To demonstrate tumor response to thermal stimulation, standard thermal images were acquired from each mouse and tumor location. Stimulated thermal imaging was also performed by heating the tumors using lasers to create a point heat source. The laser wavelengths were selected based on their predicted penetration in tissue (see FIG. 8). In these experiments, thermal stimulation was administered using blue and near-infrared (NIR) lasers. Blue lasers were quickly absorbed within the first 300 μm, generating significant heat and a strong thermal signal. In contrast, NIR lasers penetrated deeper into tissue than the blue lasers. With appropriate settings, NIR lasers may be used to stimulate tumors positioned deep within tissues. A 405 nm blue laser (fixed power output of 5 mW Gaussian beam) and a 790 nm NIR benchtop laser (BWTech, variable power output) were used to stimulate tissue for thermal imaging. The blue laser delivered laser energy to the tumor and to the contralateral side for 3-5 s each to excite thermal diffusion, followed by thermal imaging. To explore the feasibility of using LSTI to interrogate deep tumors, the mice were flipped over and the tumor side and the contralateral side were illuminated using the NIR laser, followed by thermal imaging using the same parameters as listed above.

Thermal diffusion profiles and laser spot symmetries were calculated using the thermal images obtained above. To calculate the thermal diffusion profile, each thermal image was processed using Image Pro to create a spatial temperature plot for the tumor and contralateral side. Each thermal diffusion profile was compared with a Gaussian distribution plot created using MATLAB (Mathworks). Full width at half maximum (FWHM) was used to classify whether a diffusion profile was Gaussian or non-Gaussian. Laser spot symmetry was assessed by first extracting the border of the thermal image. The spatial thermal distribution of the laser spots were plotted for the tumor and contralateral sides. Comparison of x-axis and y-axis contour dimensions was used to calculate whether the thermal distribution was symmetrical or asymmetrical.

Multiple features were extracted from the LSTI images that helped distinguish tumors from normal tissue. One of the features was the thermal diffusion profile. The heat diffusion for healthy tissue a Gaussian profile upon heating with the laser, whereas the Gaussian profile was less frequently observed in tumors. FIG. 3A is an image of a MATLAB-generated Gaussian distribution, FIG. 3B is an image of a thermal diffusion profile produced from healthy tissue, and FIG. 3C is an image of a thermal diffusion profile produced from tumor tissue. Comparing the thermal diffusion profiles of the healthy tissue (FIG. 3B) and tumor tissue (FIG. 3C) with the reference Gaussian profile (FIG. 3C) tumor-associated peak-broadening was observed.

Another LSTI feature identified was the 2D symmetry of the thermal profile (FIG. 4). Referring to FIG. 4, heat expansion on the tumor tissue (lower left spot) was detected as an asymmetric heating profile. By contrast, heat expansion on normal tissue (upper right spot) was detected as a symmetric heating profile.

To simulate deep tissue LSTI, the mice were flipped over and irradiated on the ventral side with the 405 nm laser to assess whether the 405 nm laser-generated thermal stimulation propagated through the full thickness of the mouse to reach the tumor on the dorsal side. The imaging parameters used in this experiment followed the same methods used for the dorsal imaging. Similar to the dorsal imaging, symmetry and thermal profile were investigated as features to diagnose the presence of tumors.

The symmetry and Gaussian profile features obtained for the simulated deep tumors captured from the ventral side were similar to the results for the superficial tumors captured from the dorsal side as described above. Although the ventral results were not as accurate as the results from the dorsal side, the data from the ventral measurements suggested that heat propagation to tumors may occur over several centimeters, with the Gaussian profile parameter providing the highest sensitivity (100%) and negative predictive value (100%).

### Example 3: Dye-Enhanced Deep Laser-Stimulated Thermal Imaging

To demonstrate the feasibility of using dye-enhanced deep laser-stimulated thermal imaging, the following experiments were conducted. A method of thermal recovery after laser stimulation as described in Example 2 above was used to identify cancerous tissues in mice. The mice were irradiated with a 785 nm laser on their dorsal and ventral sides, followed by thermal imaging recorded after the laser irradiation stopped. The measured thermal recovery curves indicated that the cancerous regions typically took longer to recover to steady state temperatures on both the dorsal and ventral sides as compared to healthy tissue, but this difference was not statistically significant. To enhance the thermal contrast of tumors, mice were injected with LS301, a fluorescent compound with an excitation wavelength of 785 nm and an emission wavelength of 820 nm. The measurements described above were repeated approximately 15 minutes post-injection. Tumors with LS301 uptake on the dorsal side had significantly longer thermal recovery times compared to healthy tissue. On the ventral side, thermal recovery in tumors was longer than healthy tissue.

### Example 4: Breast Cancer Treatment Response Monitoring

To assess the suitability of the LSTI method described above for monitoring of a response of breast tissue to a treatment, the following experiments were conducted. Without being limited to any particular theory, disruption of tumor vasculature, metabolism, and cell density were predicted to alter heat propagation in the tumor environment in response to a successful treatment. Tumors receiving therapies such as chemotherapy and radiation therapy likely undergo alterations to their vascular networks and metabolism under treatment and their LSTI profiles may even reflect the degree to which a tumor was responding to a therapy.

About 10⁶ PyMT-Bol murine breast cancer cells were injected into the mammary fat pad of six-week old C57BL/6 mice (60 mice). Fifteen mice in each group were treated with radiopharmaceuticals, 18F-2-fluorodeoxyglucose (FDG), titanocene (TC), and a combination of FDG+TC. Another 15 mice that did not receive any treatment served as the control group. Mice were euthanized if tumors ulcerated, altered mobility, or grew larger than 2 cm.

Mice were anesthetized with isoflurane prior to thermal imaging. A steady-state thermal image was acquired. In addition, a 405 nm laser at 5 mW was focused on a 1 mm spot and irradiated for 10 seconds prior to the acquisition of another thermal image. Imaging was performed on days 5, 8, 12, 15, 19, 22, and 26 post-injection.

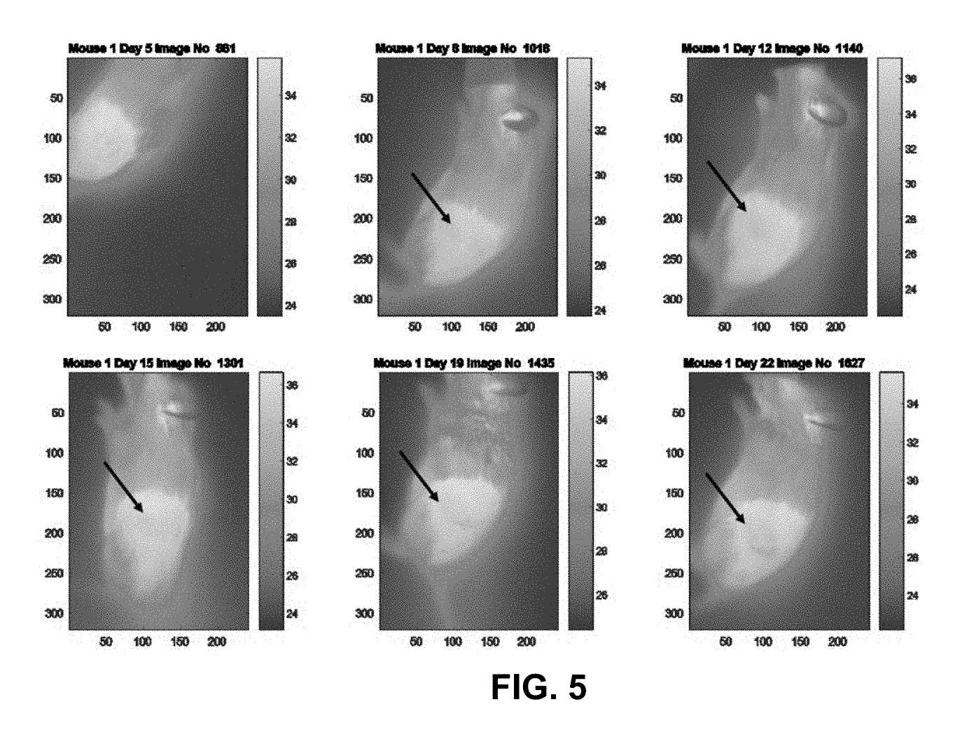

The thermal images were analyzed using methods similar to the methods described in Example 2 above. Preliminary results from the steady-state thermal images revealed that the tumors were significantly colder than the surrounding tissue, in agreement with previous studies in Balb/C mice with 4T1 tumors. FIG. 5 contains a time-series of thermal images obtained from one mouse throughout the progression of tumor growth, with the tumor highlighted by an overlaid arrow from Day 8 to Day 22 post cancer cell injection (FIG. 5).

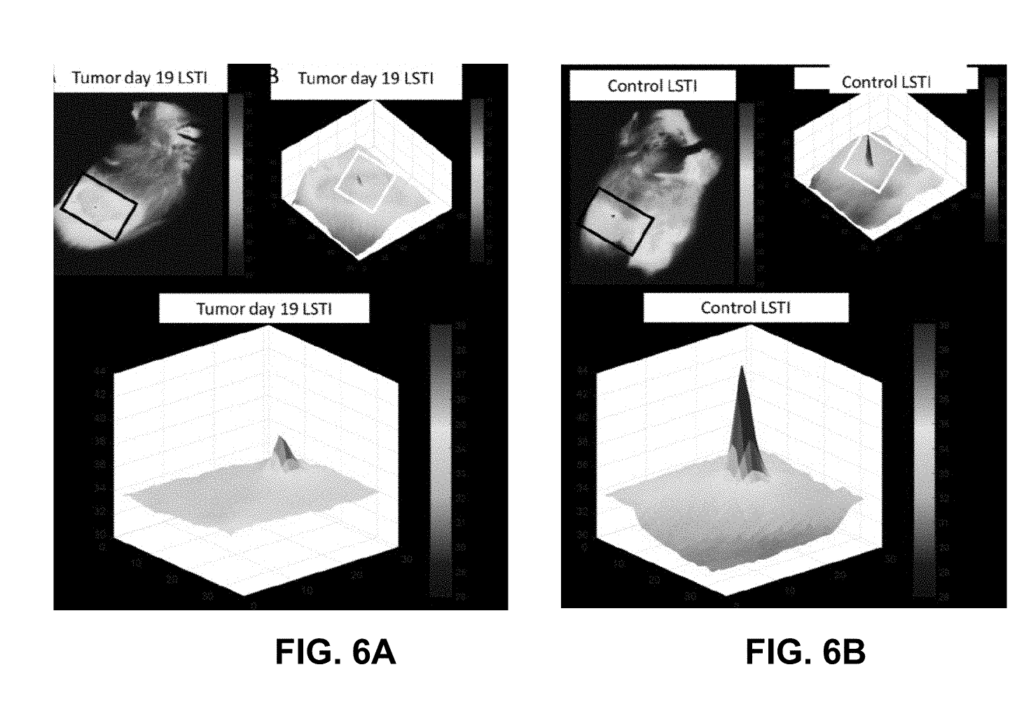

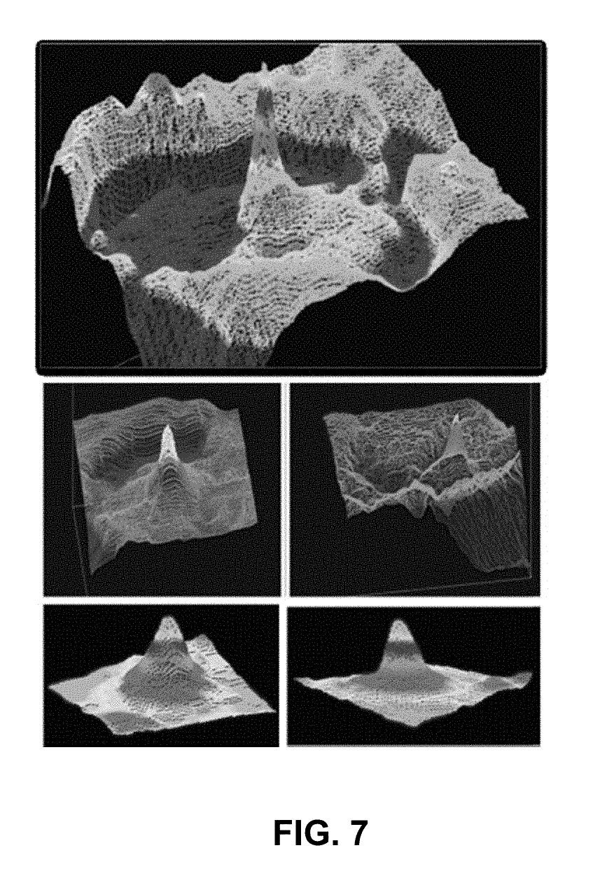

The LSTI data (FIG. 6) obtained using the model of this experiment were similar to the LSTI data obtained from balb/c 4T1 injected mice as summarized in Example 1, with tumor tissues having a broader full-width at half maximum and increased asymmetry compared to healthy tissues (FIG. 6C vs. FIG. 6F). Preliminary spatial temperature plots revealed consistent non-Gaussian peaks in laser-stimulated digitally enhanced thermal images of tumor tissues compared to healthy control tissues (FIG. 7). Further interrogation of the observed point-spread function may enable the identification of small masses and heterogeneities within and surrounding the tumor.

### Example 5: Comparison Between Computational Simulation Model and Experimental Measurements of Bacon at 5× Muscle and 5× Fat Locations

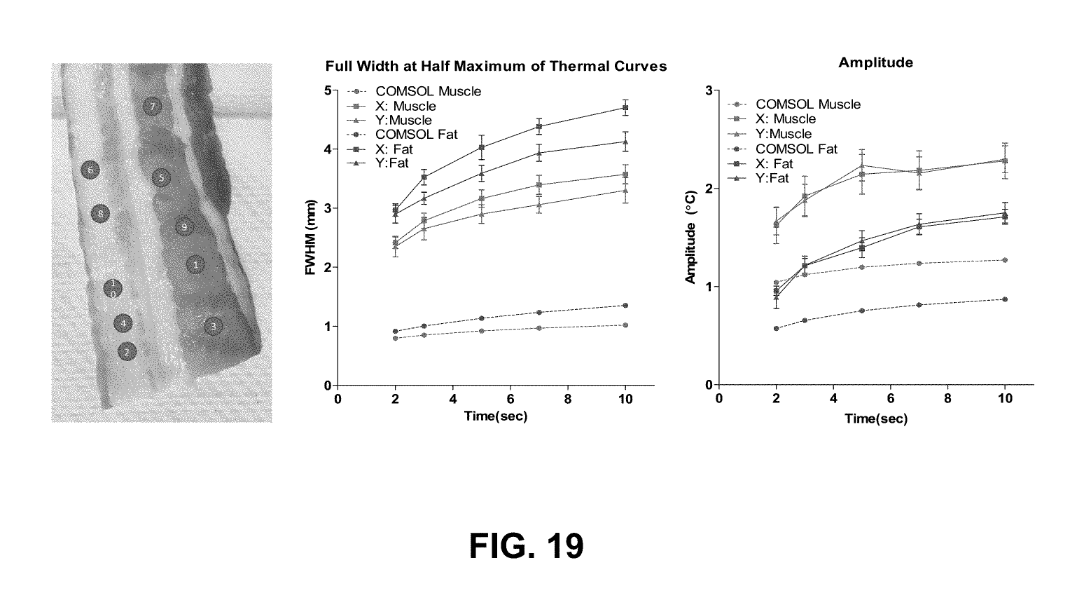

A computational simulation model was tested against experimental measurements of bacon at 5× muscle and 5× fat locations (FIG. 19). Experimental data follows the same trend as the COMSOL model, validating model. Computational simulation shows good agreement with experimental results. The LSTI technique is able to quantitatively distinguish between different biomaterials.

### Example 6: In Vivo LSTI from Rat Breast Cancer Tumors

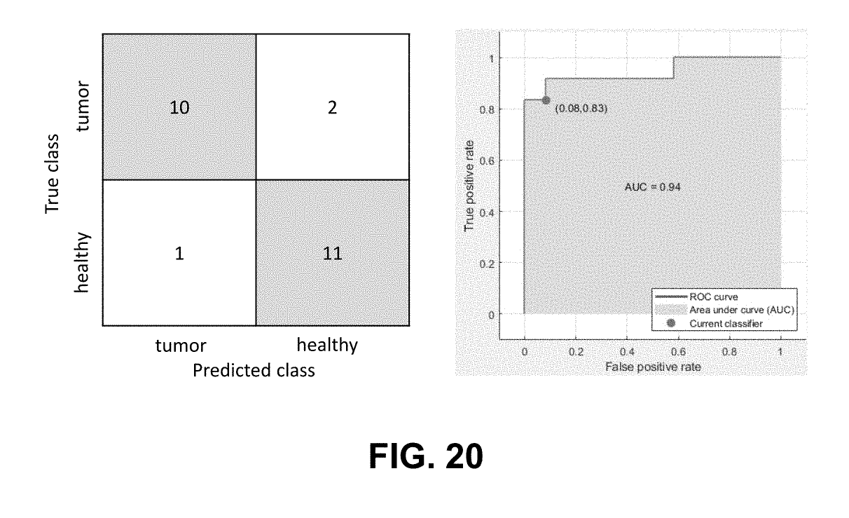

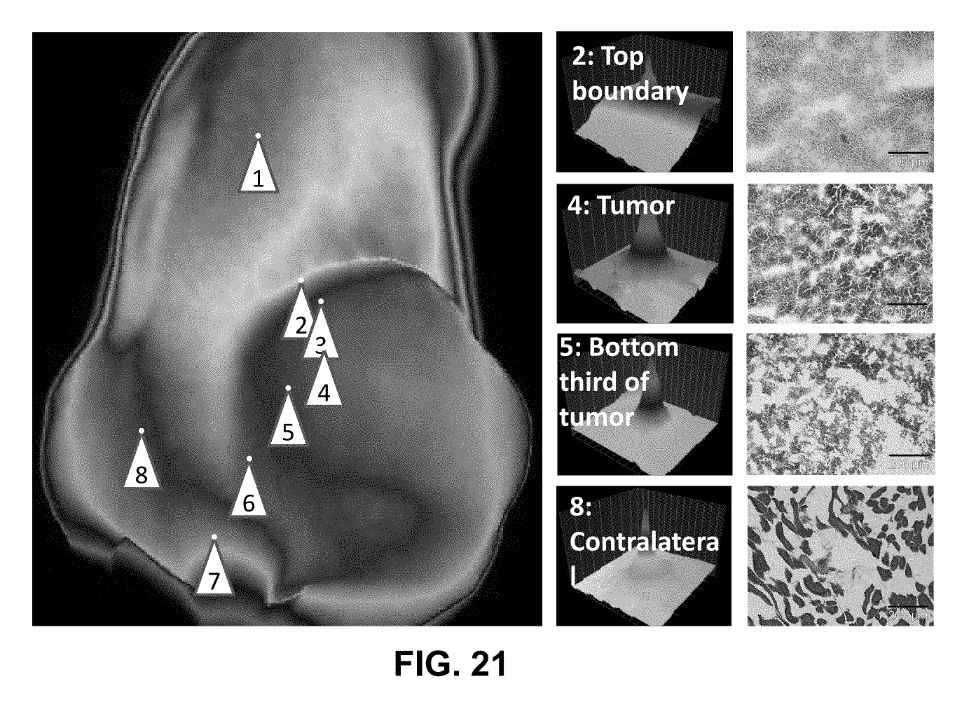

As shown in FIG. 20, 100k MAT B III mammary adenocarcinoma cells were implanted on the right flank subcutaneously in Sprague Dawley Rats, n=4. MATLAB Ensemble Subspace Discriminant Classification was used on 24 total samples with 5 fold cross-validation. The in vivo LSTI showed 94% accuracy and was superior to traditional thermal imaging. Thermal biopsy images are shown in FIG. 21.

In view of the above, it will be seen that the several advantages of the disclosure are achieved and other advantageous results attained. As various changes could be made in the above methods and systems without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

We claim the following:

1. A computer-implemented method for classifying a tissue type within a tissue sample using a laser-stimulated thermal imaging (LSTI) device communicatively coupled to a processor and a memory, the method comprising:
obtaining a first thermal image of the tissue sample;
introducing at least one thermal stimulation into at least one thermal stimulation volume within the tissue sample;
obtaining a second thermal image of the tissue sample;
creating a spatial temperature plot based on the first and second thermal images;
determining at least one thermal diffusion parameter from the spatial temperature plot; and
classifying each tissue type adjacent to each of the at least one thermal stimulation volumes based on the at least one thermal diffusion parameter.

2. The computer-implemented method of claim 1, wherein introducing at least one thermal stimulation comprises introducing at least one thermal stimulation using a single source.

3. The computer-implemented method of claim 2, wherein introducing at least one thermal stimulation using a single source comprises using one of electromagnetic radiation sources, acoustic sources, radioactive sources, chemical sources, and engineered material heat sources configured to generate or absorb heat.

4. The computer-implemented method of claim 1, wherein introducing at least one thermal stimulation comprises introducing at least one thermal stimulation using a combination of sources.

5. The computer-implemented method of claim 4, wherein introducing at least one thermal stimulation using a combination of sources comprises using a least two different sources selected from electromagnetic radiation sources, acoustic sources, radioactive sources, chemical sources, and engineered material heat sources configured to generate or absorb heat.

6. The computer-implemented method of claim 1, wherein obtaining the first and second thermal images comprises obtaining the first and second thermal images using at least one of a thermal imaging device or sensor.

7. The computer-implemented method of claim 6, wherein obtaining the first and second thermal images using at least one of a thermal imaging device or sensor comprises using at least one of thermally sensitive cameras, temperature sensors, and photon sensors.

8. A laser-stimulated thermal imaging (LSTI) system for classifying a tissue type within a tissue sample, the system comprising a LSTI device communicatively coupled to a processor and a memory, the processor programmed to:
obtain a first thermal image of the tissue sample;
introduce at least one thermal stimulation into at least one thermal stimulation volume within the tissue sample;
obtain a second thermal image of the tissue sample;
create a spatial temperature plot based on the first and second thermal images;
determine at least one thermal diffusion parameter from the spatial temperature plot; and
classify each tissue type adjacent to each of the at least one thermal stimulation volumes based on the at least one thermal diffusion parameter.

9. The LSTI system of claim 8, wherein the at least one thermal stimulation is introduced using a single source.

10. The LSTI system of claim 9, wherein the single source comprises one of electromagnetic radiation sources, acoustic sources, radioactive sources, chemical sources, and engineered material heat sources configured to generate or absorb heat.

11. The LSTI system of claim 8, wherein the at least one thermal stimulation is introduced using a combination of sources.

12. The LSTI system of claim 11, wherein the combination of sources comprises at least two sources selected from electromagnetic radiation sources, acoustic sources, radioactive sources, chemical sources, and engineered material heat sources configured to generate or absorb heat.

13. The LSTI system of claim 8, wherein the first and second thermal images are obtained using at least one of a thermal imaging device or sensor.

14. The LSTI system of claim 13, wherein the at least one thermal imaging device or sensor comprises at least one of thermally sensitive cameras, temperature sensors, and photon sensors.

15. A non-transitory computer-readable storage medium having computer-executable instructions embodied thereon, wherein when executed by a laser-stimulated thermal imaging (LSTI) system comprising a LSTI device communicatively coupled to a processor and a memory, the computer-executable instructions cause the LSTI system to:
obtain a first thermal image of the tissue sample;
introduce at least one thermal stimulation into at least one thermal stimulation volume within the tissue sample;
obtain a second thermal image of the tissue sample;
create a spatial temperature plot based on the first and second thermal images;
determine at least one thermal diffusion parameter from the spatial temperature plot; and
classify each tissue type adjacent to each of the at least one thermal stimulation volumes based on the at least one thermal diffusion parameter.

16. The non-transitory computer-readable storage media of claim 15, wherein the at least one thermal stimulation comprises using a single source or a combination of sources.

17. The non-transitory computer-readable storage media of claim 16, wherein the at least one thermal stimulation comprises electromagnetic radiation sources, acoustic sources, radioactive sources, chemical sources, and engineered material heat sources configured to generate or absorb heat.

18. The non-transitory computer-readable storage media of claim 15, wherein the first and second thermal images are obtained using at least one of a thermal imaging device or sensor.

19. The non-transitory computer-readable storage media of claim 18, wherein the at least one thermal imaging device or sensor comprises at least one of thermally sensitive cameras, temperature sensors, and photon sensors.

20. The non-transitory computer-readable storage media of claim 15, wherein the at least one thermal diffusion parameter comprises spatial thermal diffusion profiles of the tissue sample, with or without stimulation, spatial thermal hot-spot profile after stimulation of the tissue sample including Gaussian profiling, and temporal thermal profile of the tissue sample, before or after stimulation.
